(19) United States
(12) Patent Application Publication
(10) Pub. No.: US 2002/0009762 A1
(43) Pub. Date: Jan. 24, 2002

(54) Assay for protein tyrosine phosphatases

(76) Inventors: Andrew J. Flint, Bothell, WA (US); Deborah E. Cool, Snohomish, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/788,626

(22) Filed: Feb. 13, 2001

Related U.S. Application Data
(60) Provisional application No. 60/181,769, filed on Feb. 14, 2000.

(51) Int. Cl. G01N 33/53; G01N 33/537; G01N 33/543; C12Q 1/42

(52) U.S. Cl. 435/7.92; 435/4; 435/21

(57) ABSTRACT

The invention relates in part to screening assays for identifying agents that alter the interaction between a protein tyrosine phosphatase (PTP) and its tyrosine phosphorylated polypeptide substrate, using fluorescence energy signals generated by detectably labeled substrates. Assays are provided in certain embodiments, including high throughput screening assays, wherein candidate agents are screened by fluorescence polarization for their ability to influence (i) binding of substrate trapping mutant PTPs to substrates, or (ii) dephosphorylation of tyrosine phosphorylated substrates by PTPs.

### CROSS-REFERENCE TO RELATED APPLICATION

This application claims the benefit of U.S. Provisional Patent Application No. 60/181,769, filed Feb. 14, 2000, which is incorporated herein by reference in its entirety.

### TECHNICAL FIELD

The present invention relates to the protein tyrosine phosphatase family of enzymes that mediate biological signal transduction, and in particular to assays for protein tyrosine phosphatase binding to, or catalytic dephosphorylation of, tyrosine phosphorylated peptide substrates.

### BACKGROUND OF THE INVENTION

Reversible protein tyrosine phosphorylation, coordinated by the action of protein tyrosine kinases (PTKs) that phosphorylate certain tyrosine residues in polypeptides, and protein tyrosine phosphatases (PTPs) that dephosphorylate certain phosphotyrosine residues, is a key mechanism in regulating many cellular activities. It is becoming apparent that the diversity and complexity of the PTPs and PTKs are comparable, and that PTPs are equally important in delivering both positive and negative signals for proper function of cellular machinery. Regulated tyrosine phosphorylation contributes to specific pathways for biological signal transduction, including those associated with cell division, proliferation and differentiation. Defects and/or malfunctions in these pathways may underlie certain disease conditions for which effective means for intervention remain elusive, including for example, malignancy, autoimmune disorders, diabetes, obesity and infection.

The protein tyrosine phosphatase (PTP) family of enzymes consists of more than 500 structurally diverse proteins that have in common the highly conserved 250 amino acid PTP catalytic domain, but which display considerable variation in their non-catalytic segments (Charbonneau and Tonks, 1992 Annu. Rev. Cell Biol. 8:463-493; Tonks, 1993 Semin. Cell Biol. 4:373-453). This structural diversity presumably reflects the diversity of physiological roles of individual PTP family members, which in certain cases have been demonstrated to have specific functions in growth, development and differentiation (Desai et al., 1996 Cell 84:599-609; Kishihara et al., 1993 Cell 74:143-156; Perkins et al., 1992 Cell 70:225-236; Pingel and Thomas, 1989 Cell 58:1055-1065; Schultz et al.,1993 Cell 73:1445-1454). PTPs participate in a variety of physiologic functions, providing a number of opportunities for therapeutic intervention in physiologic processes through alteration or modulation (e.g., up-regulation or down-regulation) of PTP activity. For example, therapeutic inhibition of PTPs such as PTP1B in the insulin signaling pathway may serve to augment insulin action, thereby ameliorating the state of insulin resistance common in Type II diabetes patients.

Although recent studies have also generated considerable information regarding the structure, expression and regulation of PTPs, the nature of the tyrosine phosphorylated substrates through which the PTPs exert their effects remains to be determined. Studies with a limited number of synthetic phosphopeptide substrates have demonstrated some differences in the substrate selectivities of different PTPs (Cho et al., 1993 Protein Sci. 2: 977-984; Dechert et al., 1995 Eur. J Biochem. 231:673-681). Analyses of PTP-mediated dephosphorylation of PTP substrates suggest that catalytic activity may be favored by the presence of certain amino acid residues at specific positions in the substrate polypeptide relative to the phosphorylated tyrosine residue (Ruzzene et al., 1993 Eur. J. Biochem. 211:289-295; Zhang et al., 1994 Biochemistry 33:2285-2290). Thus, although the physiological relevance of the substrates used in these studies is unclear, PTPs display a certain level of substrate selectivity in vitro.

The PTP family of enzymes contains a common evolutionarily conserved segment of approximately 250 amino acids known as the PTP catalytic domain. Within this conserved domain is a unique signature sequence motif,

[I/V]HCXAGXXR[S/T)G SEQ ID NO:1,

that is invariant among all PTPs. The cysteine residue in this motif is invariant in members of the family and is known to be essential for catalysis of the phosphotyrosine dephosphorylation reaction. It functions as a nucleophile to attack the phosphate moiety present on a phosphotyrosine residue of the incoming substrate. If the cysteine residue is altered by site-directed mutagenesis to serine (e.g., in cysteine-to-serine or “CS” mutants) or alanine (e.g. cysteine-to-alanine or “CA” mutants), the resulting PTP is catalytically deficient but retains the ability to complex with, or bind, its substrate, at least in vitro.

CS mutants of certain PTP family members, for example, MKP-1 (Sun et al., 1993 Cell 75:487), may effectively bind phosphotyrosyl polypeptide substrates in vitro to form stable enzyme-substrate complexes, thereby functioning as “substrate trapping” mutant PTPs. Such complexes can be isolated from cells in which both the mutant PTP and the phosphotyrosyl polypeptide substrates are present. According to non-limiting theory, expression of such a CS mutant PTP can thus antagonize the normal function of the corresponding wildtype PTP (and potentially other PTPs and/or other components of a PTP signaling pathway) via a mechanism whereby the CS mutant binds to and sequesters the substrate, precluding substrate interaction with catalytically active, wildtype enzyme (e.g., Sun et al., 1993).

CS mutants of certain other PTP family members, however, may bind phosphotyrosyl polypeptide substrates and form complexes that exist transiently and are not stable. The CS mutant of PTP1B is an example of such a PTP. Catalytically deficient mutants of such enzymes that are capable of forming stable complexes with phophotyrosyl polypeptide substrates may be derived by mutating a wildtype protein tyrosine phosphatase catalytic domain invariant aspartate residue and replacing it with an amino acid that does not cause significant alteration of the Km of the enzyme but that results in a reduction in Kcat, as disclosed, for example, in U.S. Pat. Nos. 5,912,138 and 5,951,979, in U.S. application Ser. No. 09/323,426 and in PCT/US97/13016. For instance, mutation of Asp 181 in PTP1B to alanine to create the aspartate-to-alanine (D to A or DA) mutant PTP1B-D181A results in a PTP1B “substrate trapping” mutant enzyme that forms a stable complex with its phosphotyrosyl polypeptide substrate (e.g., Flint et al., 1997 Proc. Nat. Acad. Sci. 94:1680). Substrates of other PTPs can be identified using a similar substrate trapping approach, for example substrates of the PTP family members PTP-PEST (Garton et al., 1996 J. Mol. Cell. Biol. 16:6408), TCPTP (Tiganis et al., 1998 Mol. Cell Biol. 18:1622), PTP-HSCF (Spencer et al., 1997 J. Cell Biol. 138:845) and PTP-H1 (Zhang et al., 1999 J. Biol. Chem. 274:17806).

Currently, desirable goals for determining the molecular mechanisms that govern PTP-mediated cellular events include, inter alia, determination of PTP interacting molecules, substrates and binding partners, and identification of agents that regulate PTP activities. In some situations, however, current approaches may lead to an understanding of certain aspects of the regulation of tyrosine phosphorylation by PTPs, but still may not provide strategies to control specific tyrosine phosphorylation and/or dephosphorylation events within a cell. Accordingly, there is a need in the art for an improved ability to regulate phosphotyrosine signaling, including regulation of PTPs. An increased understanding of PTP regulation may facilitate the development of methods for modulating the activity of proteins involved in phosphotyrosine signaling pathways, and for treating conditions associated with such pathways.

Presently, a number of known screening assays for agents that regulate PTP activities are known, yet each of these assays has significant limitations in specificity, sensitivity or speed. For instance, one of the most common assays uses spectroscopic detection to measure p-nitro-phenol following hydrolysis of the simple organic phosphate ester p-nitrophenyl phosphate (pNPP). While this assay is simple to perform, it is neither specific for PTPs (pNPP is hydrolyzed by all types of phosphatases including serine/threonine phosphatases as well as PTPs), nor particularly sensitive in its detection limits. In general, because pNPP is an exceptionally poor substrate for PTPs (Zhang et al., 1994 Biochem. 33:2285) and because of the relatively poor sensitivity of typical spectroscopic detection in assays that determine pNPP hydrolysis, large quantities of PTP enzyme must be used in these assays. Such routine preparation of large amounts of a particular PTP enzyme is often impractical and/or expensive, and may further preclude adaptation of the assay to a useful high throughput screening format.

Similarly, poor specificity for PTPs is a shortcoming of assays known to the art that, through the use of fluorescence detection, exhibit improved sensitivity for detectable, hydrolyzable phosphorylated substrates relative to spectroscopic assays described above. Such fluorescence assays employ phosphate esters of fluorescein, for example OMFP (3-O-methylfluorescein phosphate, e g., Gottlin et al., 1996 J. Biol. Chem. 271:27445) or FDP (fluorescein diphosphate, e.g., Huyer et al., 1997 J. Biol. Chem. 272:843). These detectable substrates are intrinsically unstable in solution, however, making them poorly suitable for high throughput screening applications. Moreover, PTPs exhibit high specificity for phosphotyrosyl peptide substrates, as noted above, while showing poor specificity for unnatural organic phosphate esters such as OMFP or FDP. Such assays therefore suffer from unreliability due to detection of spurious phosphate group hydrolysis by contaminating phosphatases that are not PTPs, and/or inefficient hydrolysis by PTPs of the artificial organic phosphate ester substrates.

Another type of PTP assays that are known employ substrates for which PTPs have high specificity, such as tyrosine phosphorylated proteins or peptides. These assays detect PTP activity by monitoring the release of free phosphate following PTP hydrolysis of such substrates. For example, non-radioactive detection of liberated phosphate may be performed colorimetrically using malachite green reagents (Ng et al., 1994 J. Immunol. Meth. 179:177). The sensitivity of such colorimetric phosphate determination, however, is quite low. Enhanced sensitivity may be obtained in a radiometric assay of PTP-mediated dephosphorylation of a suitable tyrosine phosphorylated protein or peptide substrate by using ³²P₁-labeled substrates. Such assays, however, require frequent synthesis of new radiolabeled substrates in order to maintain the high specific radioactivity needed to obtain the desired sensitivity. These procedures become time-consuming and expensive, and involve additional procedural measures related to the storage, handling and disposal of radioactive materials. Additionally, counting radioactivity in each assay sample is a slow process, compared to the time involved in determining absorbance or fluorescence characteristics of a sample. Alternative assays that have been described for measuring PTP activity may be of limited usefulness where there is a requirement for radioactively labeled assay components and/or solid-phase immobilization of one or more assay components (see, e.g., WO 98/20024, WO 98/20156). In certain other situations, optimization of multiple assay components may be necessary, for example where distinct PTP substrates, reporter molecules and additional molecules are employed (see, e.g., WO 98/18956, WO 99/29894). Moreover, it may be difficult using existing methodologies to distinguish between (i) agents that alter (e.g., increase or decrease) PTP activity by reversible interaction with a PTP molecule (or a PTP substrate) and (ii) agents that alter PTP activity by covalently reacting with the phosphatase, for example, by modifying the side chain of the PTP catalytic domain invariant cysteine residue.

Clearly there is a need for improved assays to identify agents that regulate PTP activities. The present invention fulfills these needs and further provides other related advantages.

### SUMMARY OF THE INVENTION

According to the present invention, there are provided compositions and methods that are useful for performing screening assays to identify agents that alter PTP binding to, and PTP-mediated catalytic dephosphorylation of, phosphotyrosine peptide substrates. Thus, it is one aspect of the invention to provide a method for identifying an agent which alters the interaction between a protein tyrosine phosphatase and a tyrosine phosphorylated polypeptide which is a substrate of the protein tyrosine phosphatase, comprising contacting in the absence and in the presence of a candidate agent, a substrate trapping mutant of a protein tyrosine phosphatase and a detectably labeled tyrosine phosphorylated peptide which is a substrate of the protein tyrosine phosphatase under conditions and for a time sufficient to permit formation of a complex between the tyrosine phosphorylated peptide and the substrate trapping mutant protein tyrosine phosphatase, wherein the substrate is capable of generating a fluorescence energy signal; and comparing the fluorescence energy signal level in the absence of the agent to the fluorescence energy signal level in the presence of the agent, wherein a difference in the fluorescence energy signal level indicates the agent alters formation of a complex between the protein tyrosine phosphatase and the substrate.

In certain embodiments the fluorescence energy signal is a fluorescent polarization signal, and in certain embodiments the detectably labeled tyrosine phosphorylated peptide comprises a fluorophore, which may be fluorescein, rhodamine, Texas Red, AlexaFluor-594, AlexaFluor-488, Oregon Green, BODIPY-FL or Cy-5. In another embodiment, the substrate comprises a polypeptide sequence derived from a protein that is VCP, p130^{cas}, EGF receptor, p210 bcr:abl, MAP kinase, Shc, insulin receptor, lck or T cell receptor zeta chain. In another embodiment, the substrate trapping mutant protein tyrosine phosphatase comprises a protein tyrosine phosphatase in which the wildtype protein tyrosine phosphatase catalytic domain invariant aspartate residue is replaced with an amino acid which does not cause significant alteration of the Km of the enzyme but which results in a reduction in Kcat to less than 1 per minute. In another embodiment, the substrate trapping mutant protein tyrosine phosphatase comprises a protein tyrosine phosphatase in which the wildtype protein tyrosine phosphatase catalytic domain is mutated at an amino acid position occupied by a cysteine residue. In another embodiment the substrate trapping mutant protein tyrosine phosphatase comprises a protein tyrosine phosphatase in which at least one wildtype tyrosine residue is replaced with an amino acid that is not capable of being phosphorylated. In certain further embodiments, at least one wildtype tyrosine residue is replaced with an amino acid that is alanine, cysteine, aspartic acid, glutamine, glutamic acid, phenylalanine, glycine, histidine, isoleucine, lysine, leucine, methionine, asparagine, proline, arginine, valine or tryptophan. In another embodiment, at least one tyrosine residue that is located in a protein tyrosine phosphatase catalytic domain is replaced. In another embodiment, at least one tyrosine residue that is located in a protein tyrosine phosphatase active site is replaced. In another embodiment, the wildtype tyrosine residue is replaced with phenylalanine. In certain other embodiments, the wildtype tyrosine residue that is replaced is a protein tyrosine phosphatase conserved residue. In certain further embodiments, the conserved residue corresponds to tyrosine at amino acid position 676 in human PTPH1. In another embodiment, at least one tyrosine residue is replaced with an amino acid that stabilizes a complex comprising the protein tyrosine phosphatase and at least one substrate molecule. In another embodiment, the substrate trapping mutant protein tyrosine phosphatase is a mutated protein tyrosine phosphatase that is PTP1B, PTP-PEST, PTPγ, MKP-1, DEP-1, PTPμ, PTPX1, PTPX10, SHP2, PTP-PEZ, PTP-MEG1, LC-PTP, TC-PTP, CD45, LAR or PTPH1.

Turning to another aspect, the invention provides a method for identifying an agent which alters the interaction between a protein tyrosine phosphatase and a tyrosine phosphorylated polypeptide which is a substrate of the protein tyrosine phosphatase, comprising contacting, in the absence and in the presence of a candidate agent, a protein tyrosine phosphatase and a detectably labeled tyrosine phosphorylated peptide which is a substrate of the protein tyrosine phosphatase under conditions and for a time sufficient to permit dephosphorylation of the substrate by the protein tyrosine phosphatase, wherein the substrate is capable of generating a fluorescence energy signal; exposing the protein tyrosine phosphatase and the substrate to a reaction terminator molecule and thereby terminating dephosphorylation of the substrate; and comparing the fluorescence energy signal level of substrate which remains phosphorylated in the absence of the agent to the energy signal level of substrate which remains phosphorylated in the presence of the agent, wherein a difference in the fluorescence energy signal level indicates the agent alters the interaction between the protein tyrosine phosphatase and the substrate.

In another embodiment, the present invention provides a method of identifying an agent which alters the interaction between a protein tyrosine phosphatase and a tyrosine phosphorylated polypeptide which is a substrate of the protein tyrosine phosphatase, comprising: contacting, in the absence and in the presence of a candidate agent, a protein tyrosine phosphatase and a detectably labeled tyrosine phosphorylated peptide which is a substrate of the protein tyrosine phosphatase under conditions and for a time sufficient to permit dephosphorylation of the substrate by the protein tyrosine phosphatase, wherein the substrate is capable of generating a fluorescence energy signal; exposing the protein tyrosine phosphatase and the substrate to a reaction terminator molecule and thereby terminating dephosphorylation of the substrate; and comparing the fluorescence energy signal level of substrate which is dephosphorylated in the absence of the agent to the energy signal level of substrate which is dephosphorylated in the presence of the agent, wherein a difference in the fluorescence energy signal level indicates the agent alters the interaction between the protein tyrosine phosphatase and the substrate.

In certain embodiments, the fluorescence energy signal level is a fluorescence polarization signal level. In certain other embodiments, the detectably labeled tyrosine phosphorylated peptide substrate and the reaction terminator molecule comprise an energy transfer molecule donor-acceptor pair, and wherein the fluorescence energy signal level is a fluorescence resonance energy transfer level. In certain further embodiments, the detectably labeled tyrosine phosphorylated peptide substrate comprises an energy transfer acceptor molecule and the reaction terminator molecule comprises an energy transfer donor molecule. In certain other further embodiments, the detectably labeled tyrosine phosphorylated peptide substrate comprises an energy transfer donor molecule and the reaction terminator molecule comprises an energy transfer acceptor molecule.

In another embodiment, the reaction terminator molecule is an antibody specific for phosphotyrosine and the step of exposing is subsequent to the step of contacting the PTP and the substrate. In certain embodiments the reaction molecule is an antibody specific for a PTP substrate, an antibody specific for a PTP catalytic domain, a substrate trapping mutant protein tyrosine phosphatase, vanadate, an SH2 domain polypeptide, an IRS1 PTB domain polypeptide, an shc PH domain polypeptide or a non-antibody molecule that specifically binds to a tyrosine phosphorylated form of the detectable PTP substrate. In certain further embodiments the molecule that specifically binds to a tyrosine phosphorylated form of the detectable substrate is an SH2 domain polypeptide or a PTP-PID domain polypeptide.

It is another aspect of the invention to provide a method for determining dephosphorylation of a substrate by a protein tyrosine phosphatase, comprising contacting a protein tyrosine phosphatase and a detectable substrate of the protein tyrosine phosphatase under conditions and for a time sufficient to permit dephosphorylation of the detectable substrate by the protein tyrosine phosphatase to form a reaction mixture, wherein the detectable substrate comprises a detectably labeled tyrosine phosphorylated peptide which is a substrate of the protein tyrosine phosphatase; exposing to the reaction mixture a reaction terminator molecule; and determining the level of tyrosine phosphorylated substrate in the reaction mixture by detecting anisotropic motion of the substrate, and therefrom determining dephosphorylation of the detectable substrate by the protein tyrosine phosphatase.

In another aspect, the invention provides a method for identifying an agent that regulates dephosphorylation of a detectable substrate by a protein tyrosine phosphatase, comprising: contacting a protein tyrosine phosphatase and a detectable substrate of the protein tyrosine phosphatase under conditions and for a time sufficient to permit dephosphorylation of the detectable substrate by the protein tyrosine phosphatase in the presence of a candidate agent to form a first incomplete reaction mixture, and in the absence of a candidate agent to form a second incomplete reaction mixture, wherein the detectable substrate comprises a detectably labeled tyrosine phosphorylated peptide which is a substrate of the protein tyrosine phosphatase; exposing to each of the first and second incomplete reaction mixtures a reaction terminator molecule to form a first complete reaction mixture and a second complete reaction mixture; and comparing the level of tyrosine phosphorylated substrate in each of the first and second complete reaction mixtures by detecting anisotropic motion of the substrate, and therefrom identifying an agent that regulates dephosphorylation of the detectable substrate by the protein tyrosine phosphatase. In certain embodiments the reaction terminator molecule is an antibody specific for phosphotyrosine and the step of exposing is subsequent to the step of contacting the PTP and the substrate. In certain embodiments the reaction terminator molecule is an antibody specific for a PTP substrate, an antibody specific for a PTP catalytic domain, a substrate trapping mutant protein tyrosine phosphatase, vanadate, an SH2 domain polypeptide, an IRS1 PTB domain polypeptide, an shc PH domain polypeptide or a non-antibody molecule that specifically binds to a tyrosine phosphorylated form of the detectable PTP substrate. In certain embodiments the molecule that specifically binds to a tyrosine phosphorylated form of the detectable substrate is an SH2 domain polypeptide or a PTP-PID domain polypeptide. In certain embodiments the detectable substrate comprises a fluorophore and anisotropic motion is detected by fluorescence polarization. In certain further embodiments the fluorophore is fluorescein, rhodamine, Texas Red, AlexaFluor-594, AlexaFluor-488, Oregon Green, BODIPY-FL or Cy-5. In certain other embodiments the substrate comprises a polypeptide sequence derived from a protein that is VCP, p130^{cas}, EGF receptor, p210 bcr:abl, MAP kinase, Shc, insulin receptor, lck or T cell receptor zeta chain.

These and other aspects of the present invention will become evident upon reference to the following detailed description and attached drawings. In addition, various references are set forth herein which describe in more detail certain aspects of this invention, and are therefore incorporated by reference in their entireties.

### BRIEF DESCRIPTION OF THE DRAWINGS

FIG. 1 shows a multiple amino acid sequence alignment of the catalytic domains of various PTPs. The positions of amino acid residues of PTP1B that interact with substrate are indicated with small arrowheads, and the residue numbering at the bottom of the alignment corresponds to that for PTP1B. FIGS. 1A-1E show a multiple sequence alignment of the catalytic domains of PTPs (SEQ ID NOS:1-35). Cytosolic eukaryotic PTPs and domain 1 of RPTPs are combined into one group; domains 2 of RPTPs are in a second group and the Yersinia PTP is in a third. Invariant residues shared among all three groups are shown in lower case. Invariant and highly conserved residues within a group are shown in italics and bold, respectively. Within the Yersinia PTP sequence, residues that are either invariant or highly conserved between the cytosolic and RPTP domain sequences are in italics and bold, respectively.

FIG. 2 depicts binding of fluorescein-labeled phosphotyrosyl peptide substrates to cysteine-to-serine (CS) mutated PTP-1B, detected by fluorescence polarization.

FIG. 3 depicts binding of fluorescein-labeled phosphotyrosyl peptide substrates to aspartate-to-alanine (DA) mutated PTP-1B. detected by fluorescence polarization.

FIG. 4 shows binding curves generated by titrating candidate PTP modulators for their ability to alter binding of a fluorescein-labeled phosphotyrosyl peptide substrate to a substrate trapping mutant PTP; PTP1B-C215S (FIG. 4A); PTP1B-D181A (FIG. 4B).

FIG. 5 shows fluorescence polarization values plotted as a function of fluorescein-labeled phosphotyrosyl peptide substrate concentration.

FIG. 6 shows detection by fluorescence polarization of PTP-1B-mediated dephosphorylation of fluorescein-labeled, phosphotyrosyl EGF receptor peptide.

FIG. 7 shows detection of PTP-mediated dephosphorylation of fluorescein-labeled, phosphotyrosyl EGF receptor peptide by fluorescence polarization in a PTP catalytic assay using several different reaction terminator molecules.

FIG. 8 shows detection of PTP-mediated dephosphorylation of fluorescein-labeled, phosphotyrosyl EGF receptor peptide by fluorescence polarization in a PTP catalytic assay using several different reaction terminator molecules.

FIG. 9 shows detection of PTP-mediated dephosphorylation of fluorescein-labeled, phosphotyrosyl EGF receptor peptide by fluorescence polarization in a PTP catalytic assay using several different reaction terminator molecules.

FIG. 10 depicts the results of high throughput screening of a small molecule library for candidate PTP modulators by fluorescence polarization using a substrate trapping mutant PTP binding assay (FIG. 10A) and a PTP catalytic assay (FIG. 10B).

### DETAILED DESCRIPTION OF THE INVENTION

The present invention is directed in part to compositions and methods for monitoring and regulating PTP activity, including methods for determining catalytic dephosphorylation of protein tyrosine phosphatase substrates, and for identifying the formation of a complex between a PTP and a tyrosine phosphorylated PTP substrate. In particular, the invention relates to assays for identifying agents which alter, e.g., enhance or inhibit, the interaction between a PTP and its phosphorylated substrate. Agents identified by these assays can be agonists (e.g., agents which enhance or increase the activity of the PTP) or antagonists (e.g., agents which inhibit or decrease the activity of the PTP) of PTP activity. The agent may be an endogenous physiological substance or may be a natural or synthetic drug, including an organic small molecule as provided herein.

The invention thus pertains in part to the surprisingly sensitive and versatile screening assay configurations provided by combining PTP and fluorescence energy signal-based technologies to determine PTP-substrate complex formation and/or PTP-mediated catalytic dephosphorylation of a tyrosine phosphorylated substrate. The invention provides improved assays to identify agents that alter PTP-substrate interactions and related advantages. For example, screening assays disclosed herein may be designed to select against agents that interfere with PTP activity by chemical reactions that result in the formation of a covalent chemical bond to a PTP molecule (e.g., PTP catalytic domain invariant cysteine). Other advantages offered by the methods and compositions provided herein include screening assays for PTP-active agents that do not require the use of radionuclides, that are adaptable to high-throughput screening formats, and that do not require solid-phase immobilization of assay components or the separation of free solutes from those involved in intermolecular binding interactions.

Thus, according to certain embodiments of the present invention, PTP-specific phophotyrosine-containing peptides may be fluorescently labeled and used as substrates and/or ligands for PTPs in assays that provide simple, rapid and sensitive measurement of enzyme activity or substrate binding using fluorescence (e.g., fluorescence polarization) detection. For example, the assay of PTP enzyme catalytic activity described herein provides improved sensitivity, permitting the use of less enzyme. In this assay, the selection of phosphopeptide substrate and, in particular, the absence of a reaction terminator molecule at the initiation of the reaction (e.g., an anti-phosphotyrosine antibody; cf. WO98/18956) provide a greater maximum detectable signal while employing lower concentrations of each component. These features render the assay suitable for low cost, rapid and sensitive assays such as high throughput screening, and offer other related advantages. In certain other embodiments, the PTP binding assay of the present invention provides simplified fluorescence (e.g., fluorescence polarization) detection of PTP-substrate binding using, for example, a substrate trapping mutant PTP that may have a substituted PTP catalytic domain invariant cysteine residue (e.g., a CS mutant or a CA mutant) or a substituted PTP catalytic domain invariant aspartate residue (e.g., a DA mutant), and optionally a substituted wildtype PTP tyrosine residue. The PTP binding assays disclosed herein do not require a radioactive ligand or other certain specialized assay components such as a fusion protein and an antibody specific for the fusion protein, or a solid-phase adsorbent such as a solid-phase detection component (cf. WO98/20 156).

In certain embodiments provided herein, PTP-substrate complex formation may be determined by detecting the presence of a complex comprising a substrate trapping mutant PTP and a detectably labeled tyrosine phosphorylated peptide. This phosphopeptide is a substrate of the PTP and is capable of generating a fluorescence energy signal. As described in greater detail below, fluorescence energy signal detection, for example by fluorescence polarization, provides determination of signal levels that represent formation of a PTP-substrate molecular complex. Accordingly, and as disclosed herein, fluorescence energy signal-based comparison of PTP-substrate complex formation in the absence and in the presence of a candidate agent provides a method for identifying whether the agent alters the interaction between a PTP and its substrate.

As noted above, the invention also pertains in part to fluorescence energy signal-based determination of PTP-mediated catalytic dephosphorylation of a tyrosine phosphorylated substrate. In some embodiments, therefore, the invention relates to catalytic assays wherein a PTP and a detectably labeled tyrosine phosphorylated peptide substrate of the PTP are contacted to permit dephosphorylation of the substrate by PTP. This phosphopeptide is a substrate of the PTP and is capable of generating a fluorescence energy signal. Dephosphorylation is terminated by exposure of the enzyme-substrate reaction to a reaction terminator molecule as provided herein, and fluorescence energy signal-based determination of reactant (e.g., phosphorylated substrate) and/or product (e.g., dephosphorylated substrate) is performed. Fluorescence energy signal detection may be, for example, by fluorescence polarization or by fluorescence resonance energy transfer, or by other fluorescence methods known in the art. For instance, by way of illustration and not limitation, in certain embodiments the reaction terminator molecule forms a complex with phosphorylated (e.g., non-hydrolyzed) substrate and a level of fluorescence polarization signal generated by phosphorylated substrate is determined. In certain other embodiments, for example, the level of fluorescence polarization signal generated by dephosphorylated substrate is determined. As an example of certain other embodiments, the detectably labeled substrate and the reaction terminator molecule may comprise an energy transfer molecule donor-acceptor pair, and the level of fluorescence resonance energy transfer from energy donor to energy acceptor is determined.

As defined herein, a phosphatase is a member of the PTP family if it contains the signature motif [I/V]HCXAGXXR[S/T]G (SEQ ID NO:1). Dual specificity PTPs, i.e, PTPs which dephosphorylate both phosphorylated tyrosine and phosphorylated serine or threonine, are also suitable for use in the invention. Appropriate PTPs for use in the present invention may be any PTP family member including, but not limited to, PTP1B, PTP-PEST, PTPγ, MKP-1, DEP-1, PTPμ, PTPX1, PTPX10, SHP2, PTP-PEZ, PTP-MEG1, LC-PTP, TC-PTP, CD45, LAR and PTPH1, and mutated forms thereof.

The present invention is also directed in part to the use of substrate trapping mutant protein tyrosine phosphatases (PTPs) derived from a PTP that has been mutated in a manner that does not cause significant alteration of the Michaelis-Menten constant (Km) of the enzyme, but which results in a reduction of the catalytic rate constant (Kcat). In certain embodiments, the PTP catalytic domain invariant aspartate residue may be replaced with another amino acid. In certain other embodiments, the substrate trapping mutant PTP may be mutated by replacement of a catalytic domain cysteine residue. Under certain conditions in vivo, a PTP enzyme may itself undergo tyrosine phosphorylation in a manner that can alter interactions between the PTP and other molecules, including PTP substrates. Thus, in certain embodiments the substrate trapping mutant PTP may be further mutated by replacement of at least one tyrosine residue with an amino acid that is not capable of being phosphorylated. Substrate trapping mutant PTPs are disclosed, for example, in U.S. Pat. Nos. 5,912,138 and 5,951,979 and in U.S. application Ser. No. 09/334,575.

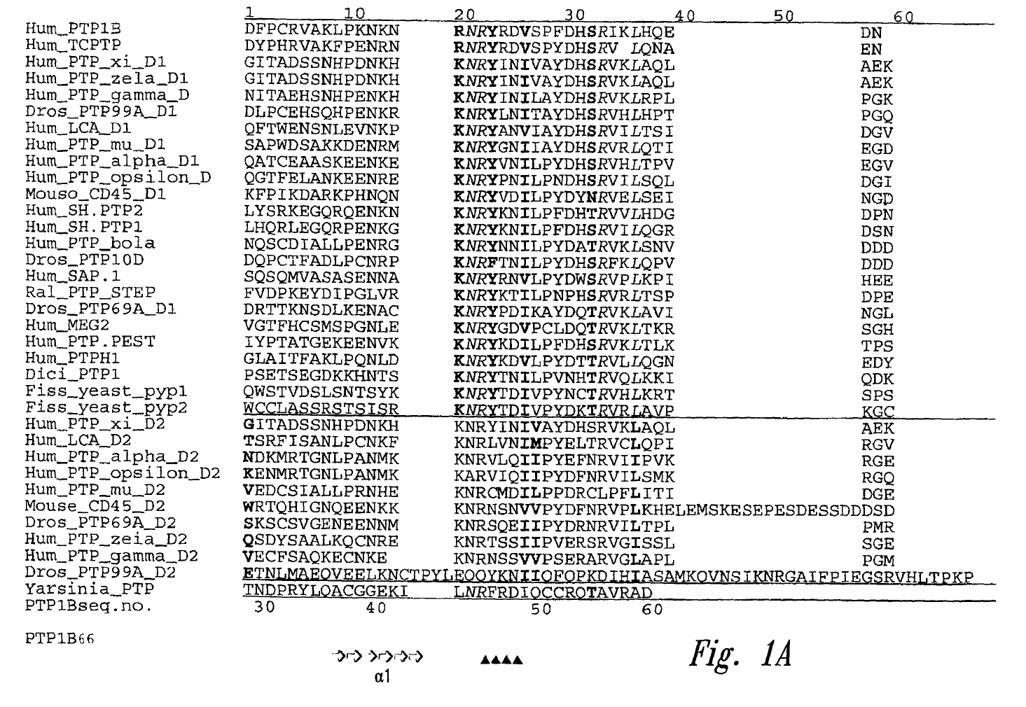

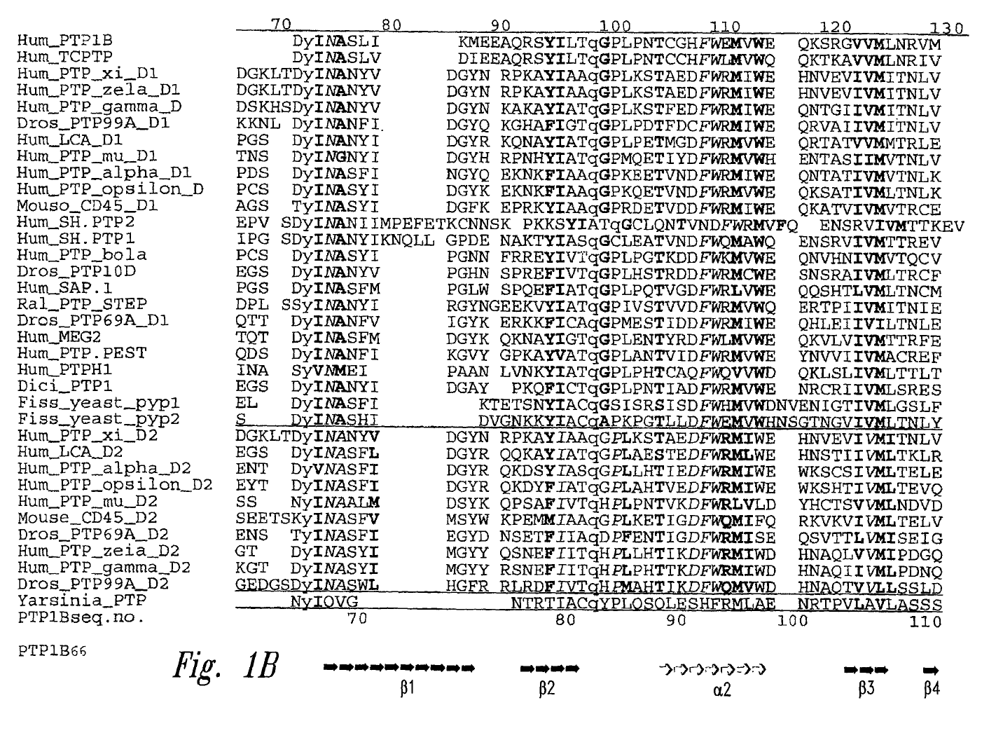

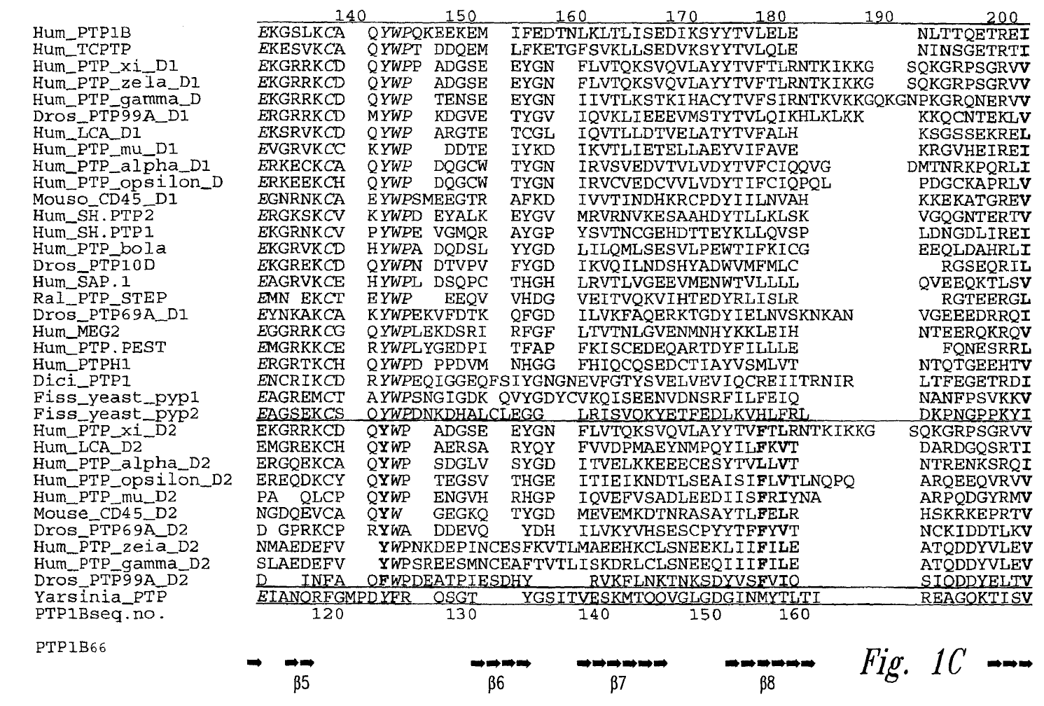

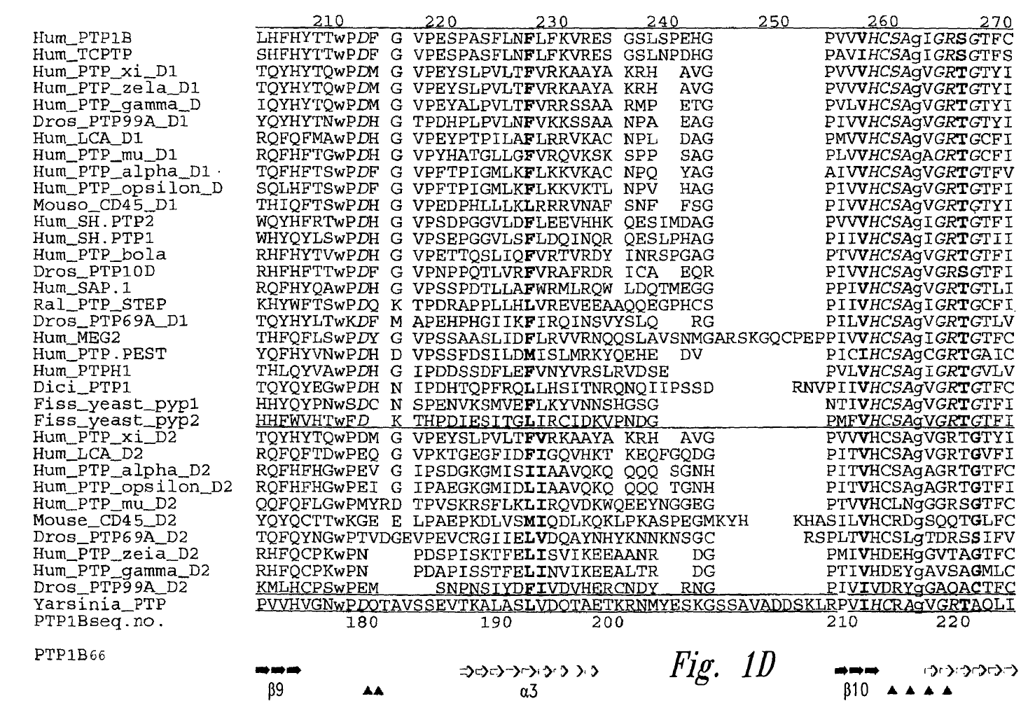

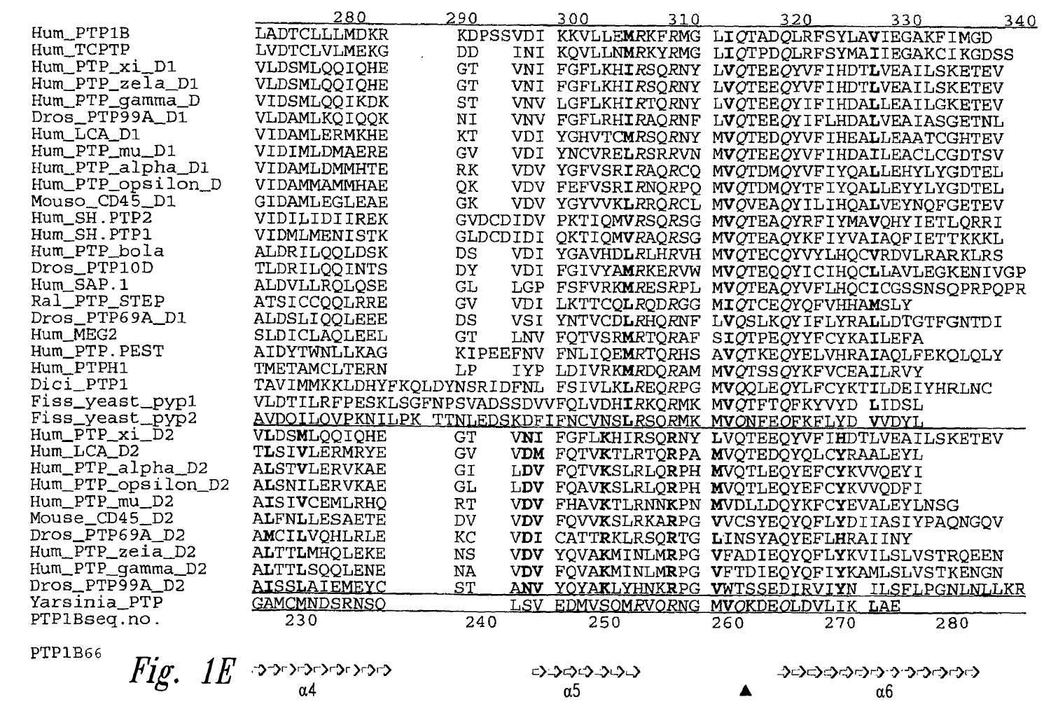

As noted above, substrate trapping mutant PTPs are derived from wildtype PTPs that have been mutated such that the wildtype protein tyrosine phosphatase catalytic domain invariant aspartate residue is replaced with an amino acid which does not cause significant alteration of the Km of the enzyme but which results in a reduction in Kcat to less than 1 per minute. Optionally, a catalytic domain cysteine residue is also replaced with a different amino acid, and/or at least one wildtype tyrosine residue is replaced with an amino acid that is not capable of being phosphorylated. In this regard, amino acid sequence analysis of known PTPs reveals the presence of twenty seven invariant residues within the PTP primary structure (Barford et al., 1994 Science 263:1397-1404; Jia et al., 1995 Science 268:1754-1758), including an aspartate residue in the catalytic domain that is invariant among PTP family members. When the amino acid sequences of multiple PTP family members are aligned (see, for instance, FIGS. 1A-E in U.S. application Ser. No. 09/334,575; see also, e.g., Barford et al., 1995 Nature Struct Biol. 2:1043), this invariant aspartate residue may be readily identified in the catalytic domain region of each PTP sequence at a corresponding position relative to the PTP signature sequence motif [I/V]HCXAGXXR[S/T]G (SEQ ID NO:2), which is invariant among all PTPs (see, e.g. WO98/04712; Flint et al., 1997 Proc. Nat. Acad. Sci. 94:1680 and references cited therein). However, the exact amino acid sequence position numbers of catalytic domain invariant aspartate residues may be different from one PTP to another, due to sequence shifts that may be imposed to maximize alignment of the various PTP sequences (see, e.g. Barford et al., 1995 Nature Struct. Biol. 2:1043 for an alignment of various PTP sequences).

In particular, portions of two PTP polypeptide sequences are regarded as “corresponding” amino acid sequences, regions, fragments or the like, based on a convention of numbering one PTP sequence according to amino acid position number, and then aligning the sequence to be compared in a manner that maximizes the number of amino acids that match or that are conserved residues, for example, that remain polar (e.g. D, E, K, R, H, S, T, N, Q), hydrophobic (e.g., A, P, V, L, I, M, F, W, Y) or neutral (e.g., C, G) residues at each position. Similarly, a DNA sequence encoding a candidate PTP that is to be mutated as provided herein, or a portion, region, fragment or the like, may correspond to a known wildtype PTP-encoding DNA sequence according to a convention for numbering nucleic acid sequence positions in the known wildtype PTP DNA sequence, whereby the candidate PTP DNA sequence is aligned with the known PTP DNA such that at least 70%, preferably at least 80% and more preferably at least 90% of the nucleotides in a given sequence of at least 20 consecutive nucleotides of a sequence are identical.

In certain preferred embodiments, a candidate PTP-encoding DNA sequence is greater than 95% identical to a corresponding known PTP-encoding DNA sequence. In certain particularly preferred embodiments, a portion, region or fragment of a candidate PTP DNA sequence is identical to a corresponding known PTP DNA sequence. As is well known in the art, an individual whose DNA contains no irregularities (e.g., a common or prevalent form) in a particular gene responsible for a given trait may be said to possess a wildtype genetic complement (genotype) for that gene, while the presence of irregularities known as mutations in the DNA for the gene, for example, substitutions, insertions or deletions of one or more nucleotides, indicates a mutated or mutant genotype.

As noted above, in certain embodiments of the present invention there is provided a substrate trapping mutant PTP in which catalytic domain invariant aspartate and at least one tyrosine residue are replaced, as provided herein. Identification of the catalytic domain invariant aspartate residue in PTP sequences other than those disclosed in Barford et al. (1995) may be achieved by comparing sequences using computer algorithms well known to those having ordinary skill in the art, such as GENEWORKS, Align or the BLAST algorithm (Altschul, J. Mol. Biol. 219:555-565, 1991; Henikoff and Henikoff, Proc. Natl. Acad. Sci. USA 89:10915-10919, 1992), which is available at the NCBI website (http://www/ncbi.nlm.nih.gov/cgi-bin/BLAST). Other sequence alignment algorithms, with which those having ordinary skill in the art will be familiar, may also be used.

Thus, substrate trapping mutant PTPs retain the ability to form a complex with, or bind to, specifically recognized tyrosine phosphorylated substrates, but are catalytically attenuated (i.e. a substrate trapping mutant PTP retains a similar Km to that of the corresponding wildtype PTP, but has a Vmax which is reduced by a factor of at least 10²-10⁵ relative to the wildtype enzyme, depending on the activity of the wildtype enzyme relative to a Kcat of less than 1 min⁻¹). This attenuation includes catalytic activity which is either reduced or abolished relative to the wildtype PTP. For example, the PTP catalytic domain invariant aspartate residue can be changed or mutated to an alanine, valine, leucine, isoleucine, proline, phenylalanine, tryptophan, methionine, glycine, serine, threonine, cysteine, tyrosine, asparagine, glutamine, lysine, arginine or histidine. In one embodiment, the invention relates to the phosphatase PTP1B in which the aspartate residue at position 181 is replaced with alanine (D181A). In another embodiment the invention relates to the phosphatase PTP-PEST in which the invariant aspartate residue at position 199 is replaced with an alanine (D199A).

The preferred substrate trapping mutant PTPs described herein, in which the invariant aspartate residue is replaced with an amino acid which does not cause significant alteration of the Km of the enzyme but which results in a reduction in Kcat to less than 1 per minute (less than 1 min⁻¹), and in which, optionally a catalytic domain cysteine residue is replaced and/or at least one tyrosine residue is replaced with an amino acid that is not capable of being phosphorylated, may further comprise other mutations. In particularly preferred embodiments, such additional mutations relate to substitutions, insertions or deletions (most preferably substitutions) that assist in stabilizing the PTP/substrate complex. For example, mutation of the serine/threonine residue in the signature motif to an alanine residue (S/T→A mutant) may change the rate-determining step of the PTP-mediated substrate dephosphorylation reaction. For the unmodified PTP, formation of the transition state may be rate-limiting, whereas in the case of the S/T→A mutant, the breakdown of the transition state may become rate-limiting, thereby stabilizing the PTP/substrate complex. Such mutations may be valuably combined with the replacement of the PTP catalytic domain invariant aspartate residue, replacement of catalytic domain cysteine and/or the replacement of PTP tyrosine as provided herein, for example, with regard to stabilizing the PTP-substrate complex and facilitating its isolation. Thus, another embodiment of the invention relates to a PTP-PEST phosphatase in which the cysteine residue at position 231 is replaced with a serine (C231S). As another example, substitution of any one or more other amino acids present in the wildtype PTP that are capable of being phosphorylated as provided herein (e.g., serine, threonine, tyrosine) with an amino acid that is not capable of being phosphorylated may be desirable, with regard to the stability of a PTP-substrate complex.

In certain other embodiments, therefore, the PTP is a mutated PTP (e.g., PTP-PEST) phosphatase in which the cysteine found in the corresponding wildtype sequence is replaced with serine and, optionally, at least one wildtype tyrosine residue is replaced with an amino acid that cannot be phosphorylated. Without wishing to be bound by any particular theory, PTPs in which wildtype cysteine residues—and in particular catalytic domain cysteines—are present, may be inactivated by candidate agents in screening assays for agents that regulate PTP binding and/or catalytic activity. In many cases, such candidate agents may be cysteine-reactive compounds and not specific PTP regulators, i.e., chemically reactive species that covalently modify cysteine and/or adjacent residues, but that do so stoichiometrically and without selectivity for PTP proteins or polypeptides. The influence of such covalent modification on PTP-substrate interactions (e.g., altered capacity for reversible complex formation, altered catalytic dephosphorylation of substrate) may therefore lead to the identification of agents that appear to alter the interaction between a PTP and its substrate, but which are in fact “false positives” that merely react with available cysteine, in particular catalytic domain cysteine. By disclosing the use of cysteine-substituted PTPs in the present invention, there is provided a method for identifying an agent which alters the interaction between a PTP and a tyrosine phosphorylated polypeptide wherein the site for non-specific covalent modification of the PTP by the agent has been eliminated.

As noted above, the present invention provides substrate trapping mutant PTPs in which catalytic domain invariant aspartate, catalytic domain cysteine and/or at least one tyrosine residue are replaced, wherein the tyrosine is replaced with an amino acid that is not capable of being phosphorylated. The amino acid that is not capable of being phosphorylated may, in preferred embodiments, be alanine, cysteine, aspartic acid, glutamine, glutamic acid, phenylalanine, glycine, histidine, isoleucine, lysine, leucine, methionine, asparagine, proline, arginine, valine or tryptophan. The desirability of the tyrosine replacement derives from the observation that under certain conditions in vivo, a PTP enzyme may itself undergo tyrosine phosphorylation in a manner that can alter interactions between the PTP and other molecules, including PTP substrates (Zhang et al., 1999 J. Biol. Chem. 274:17806). Thus, replacement of a tyrosine residue found in the wildtype amino acid sequence of a particular PTP with another amino acid as provided herein stabilizes a complex formed by the subject invention substrate trapping mutant PTP and a PTP substrate when the amount of complex that is present and/or the affinity of the mutant PTP for the substrate increases, relative to complex formation using a PTP in which the tyrosine residue is not replaced.

Preferably the tyrosine residue is located in the PTP catalytic domain, which refers to the approximately 250 amino acid region that is highly conserved among the various PTPs, as noted above (see also, e.g. Barford, 1998 Ann. Rev. Biophys. Biomol. Struct 27:133; Jia, 1997 Biochem. Cell Biol. 75:17; Van Vactor et al., 1998 Curr. Opin Genet. Devel. 8:112) More preferably, the tyrosine residue is located in a PTP active site, which refers to the region within the PTP catalytic domain that contains the PTP signature motif and which also includes those amino acids that form the PTP binding site pocket or “cradle” for substrate binding and dephosphorylation, further including the invariant aspartate-containing loop (when present) and adjacent peptide backbone sequences that contribute to substrate recognition and catalysis (see, e.g., Jia, 1997). In other preferred embodiments, the tyrosine residue is a PTP conserved residue, which includes tyrosine residues that are present at corresponding positions within two or more PTP amino acid sequences relative to the position of the signature motif. In other preferred embodiments, the tyrosine residue is replaced with an amino acid that stabilizes a complex formed by the PTP and at least one substrate molecule, as provided herein.

In a most preferred embodiment, the tyrosine residue is replaced with phenylalanine, and in another most preferred embodiment, the tyrosine residue is a conserved residue that corresponds to the tyrosine situated at position 676 in the amino acid sequence of human PTPH1, and which also corresponds to the amino acid residue at position 46 in the PTP-1B sequence (see, e.g., FIG. 1). Hence, in certain particularly preferred embodiments, the PTP is PTPH1 having the invariant aspartate replaced by alanine and the tyrosine at position 676 replaced by phenylalanine, PTPH1(Y676F/D811A).

It should be recognized, however, that mutant PTPs other than those specifically described herein can readily be made by aligning the amino acid sequence of a PTP catalytic domain with the amino acid sequence of PTPs that are described herein (including those provided by the cited references), identifying the catalytic domain invariant aspartate residue and, optionally, at least one catalytic domain cysteine residue and/or at least one tyrosine residue, and introducing amino acid substitutions at these residues, for example by site-directed mutagenesis of DNA encoding the PTP.

Modification of DNA may be performed by a variety of methods, including site-specific or site-directed mutagenesis of DNA encoding the PTP and the use of DNA amplification methods using primers to introduce and amplify alterations in the DNA template, such as PCR splicing by overlap extension (SOE). Site-directed mutagenesis is typically effected using a phage vector that has single- and double-stranded forms, such as M13 phage vectors, which are well-known and commercially available. Other suitable vectors that contain a single-stranded phage origin of replication may be used (see, e g., Veira et al., Meth. Enzymol. 15:3, 1987). In general, site-directed mutagenesis is performed by preparing a single-stranded vector that encodes the protein of interest (e.g., a member of the PTP family). An oligonucleotide primer that contains the desired mutation within a region of homology to the DNA in the single-stranded vector is annealed to the vector followed by addition of a DNA polymerase, such as E. Coli DNA polymerase I (Klenow fragment), which uses the double stranded region as a primer to produce a heteroduplex in which one strand encodes the altered sequence and the other the original sequence. Additional disclosure relating to site-directed mutagenesis may be found, for example, in Kunkel et al. (Methods in Enzymol 154:367, 1987); and in U.S. Pat. Nos. 4,518,584 and 4,737,462. The heteroduplex is introduced into appropriate bacterial cells, and clones that include the desired mutation are selected. The resulting altered DNA molecules may be expressed recombinantly in appropriate host cells to produce the modified protein.

Specific substitutions of individual amino acids through introduction of site-directed mutations are well-known and may be made according to methodologies with which those having ordinary skill in the art will be familiar. The effects on catalytic activity of the resulting mutant PTP may be determined empirically merely by testing the resulting modified protein for the preservation of the Km and reduction of Kcat to less than 1 per minute as provided herein and as previously disclosed (e.g., WO98/04712; Flint et al., 1997 Proc. Nat. Acad. Sci. 94:1680). The effects on the ability to tyrosine phosphorylate the resulting mutant PTP molecule can also be determined empirically merely by testing such a mutant for the presence of phosphotyrosine, as also provided herein, for example, following exposure of the mutant to conditions in vitro or in vivo where it may act as a PTK acceptor.

Although specific examples of PTP mutants described herein include DA (aspartate to alanine) mutants, YF (tyrosine to phenylalanine) mutants, CS mutants and combinations thereof it will be understood that the subject invention substrate trapping mutant PTPs are not limited to these amino acid substitutions. The invariant aspartate residue can be changed, for example by site-directed mutagenesis, to any amino acid that does not cause significant alteration of the Km of the enzyme but which results in a reduction in Kcat to less than 1 per minute (less than 1 min⁻¹). For example, the invariant aspartate residue can be changed or mutated to an alanine, valine, leucine, isoleucine, proline, phenylalanine, tryptophan, methionine, glycine, serine, threonine, cysteine, tyrosine, asparagine, glutamine, lysine, arginine or histidine, or other natural or non-natural amino acids known in the art including derivatives, variants and the like. Similarly, substitution of at least one tyrosine residue may be with any amino acid that is not capable of being phosphorylated (i.e., stable, covalent modification of an amino acid side chain at a hydroxyl with a phosphate group), for example alanine, cysteine, aspartic acid, glutamine, glutamic acid, phenylalanine, glycine, histidine, isoleucine, lysine, leucine, methionine, asparagine, proline, arginine, valine or tryptophan, or other natural or non-natural amino acids known in the art including derivatives, variants and the like.

Substrates

In preferred embodiments, a PTP substrate may be any naturally or non-naturally tyrosine-phosphorylated peptide, polypeptide or protein that can specifically bind to and/or be dephosphorylated by a PTP as provided herein. Non-limiting examples of known PTP substrates include the proteins VCP (see, e.g., Zhang et al., 1999 J. Biol. Chem. 274:17806, and references cited therein), p130^{cas}, EGF receptor, p210 bcr:abl, MAP kinase, Shc (Tiganis et al., 1998 Mol. Cell. Biol. 18:1622-1634), insulin receptor, lck (lymphocyte specific protein tyrosine kinase, Marth et al., 1985 Cell 43:393), and T cell receptor zeta chain. As another example, tyrosine phosphorylated peptides identified with mutant PTPs from peptide libraries by the methods of Songyang et al. (1995 Nature 373:536-539; 1993 Cell 72:767-778) can be used herein in place of the complete tyrosine phosphorylated protein in PTP binding and/or catalytic assays. Optionally, candidate peptide sequences may be selected and optimized for dephosphorylation or binding activity as described herein using other techniques such as affinity selection followed by mass spectrometric detection (e.g., Pellegrini et al., 1998 Biochem. 37:15598; Huyer et al., 1998 Anal. Biochem. 258:19) or by “inverse alanine scanning” (e.g., Vetter et al., 2000 J. Biol. Chem. 275:2265). In certain particularly preferred embodiments, a PTP substrate is a tyrosine phosphorylated peptide, which may include a partial amino acid sequence, portion, region, fragment, variant, derivative or the like from a naturally or non-naturally tyrosine-phosphorylated peptide, polypeptide or protein that can specifically bind to and/or be dephosphorylated by a PTP. A PTP substrate that is a tyrosine phosphorylated peptide typically comprises 2-100 amino acids, preferably 2-50 amino acids, more preferably 2-25 amino acids, still more preferably 2-15 amino acids and most preferably 2-10 amino acids. In certain other embodiments, a PTP substrate may comprise a phosphotyrosine residue having an attached fluorescent label.

Identification and selection of PTP substrates as provided herein, for use in the present invention, may be performed according to procedures with which those having ordinary skill in the art will be familiar, or may, for example, be conducted according to the disclosure of U.S. application Ser. No. 09/334,575 and references cited therein. The phosphorylated protein/PTP complex may be isolated, for example, by conventional isolation techniques as described in U.S. Pat. No. 5,352,660, including salting out, chromatography, electrophoresis, gel filtration, fractionation, absorption, polyacrylamide gel electrophoresis, agglutination, combinations thereof or other strategies. PTP substrates that are known may also be prepared according to well known procedures that employ principles of molecular biology and/or peptide synthesis (e.g., Ausubel et al., 1993 Current Protocols in Molecular Biology, Greene Publ. Assoc. Inc. & John Wiley & Sons, Inc., Boston, Mass.; Sambrook et al., 1989 Molecular Cloning, Second Ed., Cold Spring Harbor Laboratory, Plainview, N.Y.; Fox, 1995 Molec. Biotechnol. 3:249; Maeji et al., 1995 Pept. Res. 8:33).

The PTP substrate peptides of the present invention may therefore be derived from PTP substrate proteins, polypeptides and peptides as provided herein having amino acid sequences that are identical or similar to tyrosine phosphorylated PTP substrate sequences known in the art. For example by way of illustration and not limitation, peptide sequences derived from the known PTP substrate proteins referred to above are contemplated for use according to the instant invention, as are peptides having at least 70% similarity (preferably 70% identity), more preferably 90% similarity (more preferably 90% identity) and still more preferably 95% similarity (still more preferably 95% identity) to the polypeptides described in references cited herein and in the Examples and to portions of such polypeptides as disclosed herein. As known in the art “similarity” between two polypeptides is determined by comparing the amino acid sequence and conserved amino acid substitutes thereto of the polypeptide to the sequence of a second polypeptide (e.g., using GENEWORKS, Align or the BLAST algorithm, or another algorithm, as described above).

Thus, according to the present invention, substrates may include full length tyrosine phosphorylated proteins and polypeptides as well as fragments (e.g. portions), derivatives or analogs thereof that can be phosphorylated at a tyrosine residue. Such fragments, derivatives and analogs include any PTP substrate polypeptide that retains at least the biological function of interacting with a PTP as provided herein, for example by forming a complex with a PTP and/or, in certain embodiments, undergoing PTP-catalyzed dephosphorylation. A fragment, derivative or analog of a peptide, protein or polypeptide as provided herein, including a PTP substrate polypeptide, and further including PTP substrates that are fusion proteins, may be (i) one in which one or more of the amino acid residues are substituted with a conserved or non-conserved amino acid residue (preferably a conserved amino acid residue), and such substituted amino acid residue may or may not be one encoded by the genetic code, or (ii) one in which one or more of the amino acid residues includes a substituent group, or (iii) one in which the substrate polypeptide is fused with another compound, such as a compound to increase the half-life of the polypeptide (e.g., polyethylene glycol) or a detectable moiety such as a reporter molecule, or (iv) one in which additional amino acids are fused to the substrate polypeptide, including amino acids that are employed for purification of the substrate polypeptide or a proprotein sequence. Such fragments, derivatives and analogs are deemed to be within the scope of those skilled in the art.

In preferred embodiments the PTP substrate is detectably labeled, and in particularly preferred embodiments the PTP substrate is capable of generating a fluorescence energy signal. The PTP substrate can be detectably labeled by covalently or non-covalently attaching a suitable reporter molecule or moiety, for example any of various fluorescent materials (e.g., a fluorophore) selected according to the particular fluorescence energy technique to be employed, as known in the art and based upon the present disclosure. Fluorescent reporter moieties and methods for labeling PTP substrates as provided herein can be found, for example in Haugland (1996 Handbook of Fluorescent Probes and Research Chemicals-Sixth Ed., Molecular Probes, Eugene, O R; 1999 Handbook of Fluorescent Probes and Research Chemicals-Seventh Ed., Molecular Probes, Eugene, O R, http://www.probes.com/lit/) and in references cited therein. Particularly preferred for use as such a fluorophore in the subject invention methods are fluorescein, rhodamine, Texas Red, AlexaFluor-594, AlexaFluor-488, Oregon Green, BODIPY-FL or Cy-5.

Fluorescence Energy Signals

As provided herein, a fluorescence energy signal includes any fluorescence emission, excitation, energy transfer, quenching or dequenching event or the like. Typically a fluorescence energy signal may be mediated by a fluorescent detectably labeled tyrosine phosphorylated PTP substrate peptide in response to light of an appropriate wavelength. Briefly, and without wishing to be bound by theory, generation of a fluorescence energy signal generally involves excitation of a fluorophore (e.g., a fluorescent detectably labeled PTP substrate) by an appropriate energy source (e.g., light of a suitable wavelength for the selected fluorescent reporter moiety, or fluorophore) that transiently raises the energy state of the fluorophore from a ground state to an excited state. The excited fluorophore in turn emits energy in the form of detectable light typically having a different (e.g., usually longer) wavelength from that used for excitation, and in so doing returns to its energetic ground state. The methods of the present invention contemplate the use of any fluorescence energy signal, depending on the particular fluorophore, substrate labeling method and detection instrumentation, which may be selected readily and without undue experimentation according to criteria with which those having ordinary skill in the art will be familiar.

In certain particularly preferred embodiments of the instant invention, the fluorescence energy signal is a fluorescence polarization (FP) signal. In certain other particularly preferred embodiments of the present invention, the fluorescence energy signal may be a fluorescence resonance energy transfer (FRET) signal. In certain other preferred embodiments the fluorescence energy signal can be a fluorescence quenching (FQ) signal or a fluorescence resonance spectroscopy (FRS) signal. (For details regarding FP, FRET, FQ and FRS, see, for example, WO97/39326; WO099/29894; Haugland, Handbook of Fluorescent Probes and Research Chemicals-6th Ed., 1996, Molecular Probes, Inc., Eugene, O R, p. 456; and references cited therein.)

PTP Binding Assay

As noted above, the present invention exploits the substrate trapping mutant PTPs described herein to provide a method of identifying an agent which alters the interactions between a PTP and a tyrosine phosphorylated polypeptide which is a substrate of the PTP. The binding interaction between a PTP and a PTP substrate may result in the formation of a complex, which refers to a specific intermolecular association that results from an affinity interaction between a PTP and a suitable PTP substrate as provided herein.

A PTP-substrate complex may be identified by any of a variety of techniques known in the art for demonstrating an intermolecular interaction between a PTP and a PTP substrate as described above, for example, co-purification, co-precipitation, co-immunoprecipitation, radiometric or fluorimetric assays, western immunoblot analyses, affinity capture including affinity techniques such as solid-phase ligand-counterligand sorbent techniques, affinity chromatography and surface affinity plasmon resonance, and the like (see, e.g. U.S. Pat. No. 5,352,660). Determination of the presence of a PTP/substrate complex may employ antibodies, including monoclonal, polyclonal, chimeric and single-chain antibodies, and the like, that specifically bind to the PTP or the tyrosine phosphorylated protein substrate.

Labeled PTPs and/or labeled tyrosine phosphorylated substrates can also be used to detect the presence of a complex. The PTP or phosphorylated protein can be labeled by covalently or non-covalently attaching a suitable reporter molecule or moiety, for example any of various enzymes, fluorescent materials, luminescent materials and radioactive materials. Examples of suitable enzymes include, but are not limited to, horseradish peroxidase, biotin, alkaline phosphatase, β-galactosidase and acetylcholinesterase. Examples of suitable fluorescent materials include, but are not limited to, umbelliferone, fluorescein, fluorescein isothiocyanate, rhodamine, dichlorotriazinylamine fluorescein, dansyl chloride, phycoerythrin, Texas Red, AlexaFluor-594, AlexaFluor-488, Oregon Green. BODIPY-FL and Cy-5. Appropriate luminescent materials include luminol, and suitable radioactive materials include radioactive phosphorus [³²P], iodine [¹²⁵I or ¹³¹I] or tritium [³H].

According to the subject invention, at least one substrate trapping mutant PTP as provided herein is combined with at least one detectably labeled tyrosine phosphorylated polypeptide which is a substrate of the PTP, wherein the substrate (e.g. a substrate identified as described above) is capable of generating a fluorescence energy signal. The mutant PTP and the substrate are combined under conditions and for a time sufficient to permit formation of an intermolecular complex between the PTP and the substrate. Suitable conditions for formation of such complexes are known in the art and can be readily determined based on teachings provided herein, including solution conditions and methods for detecting the presence of a complex and/or for detecting free substrate in solution.

Association of a detectably labeled substrate in a complex with a substrate trapping mutant PTP, and/or free substrate that is not part of such a complex, may be identified according to the present invention by detection of a fluorescence energy signal generated by the substrate. Typically, an energy source for detecting a fluorescence energy signal is selected according to criteria with which those having ordinary skill in the art will be familiar, depending on the fluorescent reporter moiety with which the substrate is labeled. The test solution, containing (a) the mutant PTP and (b) the detectably labeled PTP substrate, is exposed to the energy source to generate a fluorescence energy signal, which is detected by any of a variety of well known instruments identified according to the particular fluorescence energy signal. In preferred embodiments, the fluorescence energy signal is a fluorescence polarization signal that can be detected using a spectrofluorometer equipped with polarizing filters. In particularly preferred embodiments the fluorescence polarization assay is performed simultaneously in each of a plurality of reaction chambers that can be read using an LJL CRITERION™ Analyst (LJL Biosystems, Sunnyvale, Calif.) plate reader, for example, to provide a high throughput screen (HTS) having varied reaction components or conditions among the various reaction chambers. Examples of other suitable instruments for obtaining fluorescence polarization readings include the PolarStar™ (BMG Lab Technologies, Offenburg, Germany), Beacon™ (Panvera, Inc., Madison, Wis.) and the Polarion™ (Tecan, Inc., Research Triangle Park, N.C.) devices.

Determination of the presence of a complex that has formed between a PTP and a PTP substrate refers to the use of any fluorescence energy signal methodology as provided herein and as known in the art for demonstrating an intermolecular interaction between a PTP and a PTP substrate according to the present disclosure. Such methodologies may include, by way of illustration and not limitation FP, FRET, FQ and other fluorimetric assays. In preferred embodiments, the presence or absence of a complex is determined by FP. A PTP may interact with a PTP substrate via specific binding if the PTP binds the substrate with a Kₐ of greater than or equal to about 10⁴ M⁻¹, preferably of greater than or equal to about 10⁵ M⁻¹, more preferably of greater than or equal to about 10⁶ M⁻¹ and still more preferably of greater than or equal to about 10⁷ M⁻¹ to 10¹¹ M⁻¹. Affinities of binding partners such as a PTP and a PTP substrate can be readily calculated from data generated according to the fluorescence energy signal methodologies described above and using conventional data handling techniques, for example, those described by Scatchard et al., Ann. N. Y Acad. Sci. 51:660 (1949).

For example, in preferred embodiments where the fluorescence energy signal is a fluorescence polarization signal, fluorescence anisotropy (in polarized light) of the free detectably labeled PTP substrate peptide can be determined in the absence of substrate trapping PTP (i.e. greatest freedom of rotation because no labeled peptide is involved in complex formation), and fluorescence anisotropy (in polarized light) of the fully bound substrate can be determined in the presence of a titrated amount of the substrate trapping PTP (i.e. a PTP concentration where all substrate is complexed to mutant PTP the fluorophore will exhibit the least freedom of rotation). Fluorescence anisotropy in polarized light varies as a function of the amount of rotational motion that the labeled substrate undergoes during the lifetime of the excited state of the fluorophore, such that the anisotropies of free and fully bound substrate can be usefully employed to determine the fraction of substrate bound to PTP in a given set of experimental conditions, for instance, those wherein a candidate agent is present (see, e.g., Lundblad et al., 1996 Molec. Endocrinol. 10:607; Dandliker et al., 1971 Immunochem. 7:799; Collett, E., Polarized Light: Fundamentals and Applications, 1993 Marcel Dekker, New York).

FP, a measurement of the average angular displacement (due to molecular rotational diffusion) of a fluorophore that occurs between its absorption of a photon from an energy source and its subsequent emission of a photon, depends on the extent and rate of rotational diffusion during the excited state of the fluorophore, on molecular size and shape, on solution viscosity and on solution temperature (Perrin, 1926 J. Phys. Rad. 1:390). When viscosity and temperature are held constant, FP is directly related to the apparent molecular volume or size of the fluorophore. The polarization value is a ratio of fluorescence intensities measured in distinct planes (e.g., vertical and horizontal) and is therefore a dimensionless quantity that is unaffected by the intensity of the fluorophore. Low molecular weight fluorophores, such as the detectably labeled PTP substrate polypeptides provided herein, are capable of rapid molecular rotation in solution (i.e., low anisotropy) and thus give rise to low fluorescence polarization readings. When complexed to a higher molecular weight molecule such as a substrate trapping mutant PTP as provided herein, however, the fluorophore moiety of the substrate associates with a complex that exhibits relatively slow molecular rotation in solution (i.e., high anisotropy), resulting in higher fluorescence polarization readings.

This difference in the polarization value of free detectably labeled PTP substrate polypeptide compared to the polarization value of a substrate trapping PTP mutant:substrate complex may be used to determine the ratio of complexed (e.g. bound) substrate to free substrate. This difference may also be used to detect the influence of a candidate agent on the formation of such complexes and/or on the stability of a pre-formed complex, for example by comparing FP detected in the absence of an agent to FP detected in the presence of the agent. FP measurements can be performed without separation of reaction components, e.g., PTP-bound substrate need not be separated from free substrate.

For example, if a candidate agent competitively binds to either the substrate trapping mutant PTP or the PTP substrate polypeptide in a manner that precludes formation of a substrate trapping PTP mutant:substrate complex, the detectably labeled substrate remains free in solution and decreased (i.e., altered) polarization readings are obtained. As another example, if a candidate agent fails to competitively inhibit or promotes substrate trapping PTP mutant:substrate complex formation. substrate-bound fluorophore remains in the complex and either unchanged or increased (i.e., altered) polarization readings are observed.

Without wishing to be bound by theory, it is contemplated that phosphorylated tyrosine residues that are part of a PTP molecule itself may influence the interaction between the PTP molecule and PTP substrate molecules, which include tyrosine phosphorylated proteins that a PTP may bind and/or dephosphorylate. According to this non-limiting theory, a conserved tyrosine residue present in a PTP primary structure may be a receptor for transfer of a phosphate group from the phosphate group present in the form of phosphotyrosine on the PTP substrate phosphoprotein (Zhang et al., 1999 J. Biol. Chem. 274:17806). Thus, although a conserved tyrosine residue in a PTP active site may facilitate intermolecular orientation of the PTP relative to its substrate by providing a hydrophobic interaction with the substrate phosphotyrosine, and may further act as a phosphate acceptor, the invention is not so limited.

As noted above, the present invention relates in part to a method of identifying an agent which alters the interaction between a PTP and a tyrosine phosphorylated polypeptide which is a substrate of the PTP. The method comprises contacting, in the absence and presence of a candidate agent, a substrate trapping PTP and a detectably labeled tyrosine phosphorylated peptide which is a substrate of the PTP under conditions conducive to formation of a PTP-substrate complex, wherein the substrate is capable of generating a fluorescence energy signal as provided herein. The fluorescence energy signal level detected in the absence of the candidate agent is compared to the level in the presence of the agent, such that a difference in signal level indicates that the agent alters PTP-substrate complex formation. As described above, in certain embodiments the substrate trapping PTP comprises a PTP in which the invariant aspartate residue is replaced with an amino acid which does not cause significant alteration of the Km of the enzyme but which results in a reduction in Kcat to less than 1 per minute (less than 1 mind) (e.g., an alanine residue). In certain other embodiments the substrate trapping PTP may comprise a CS mutant PTP as described above, which is catalytically deficient but remains capable of forming a stable or unstable complex in solution with a phosphotyrosine peptide substrate. Thus, the substrate trapping PTP mutant binds to or complexes with its substrate but does not dephosphorylate it (or does so very slowly), thereby allowing the level of complex formation and/or the level of free substrate to be detected.

For example, a competitive binding assay can be carried out utilizing the mutant PTP in the presence of an agent to be tested, and the resulting extent of binding of the mutant PTP to its substrate can be compared with the extent of binding in the absence of the agent to be tested. A decrease in the extent of binding in the presence of the agent to be tested indicates that the agent inhibits the interaction between the PTP and its substrate. Conversely, an increase in the extent of binding in the presence of the agent to be tested indicates that the agent enhances the interaction between the PTP and its substrate.

As described herein, the binding affinity for substrate of mammalian protein tyrosine phosphatases (PTPs) may be determined using fluorescence polarization (FP) detection in concert with a substrate trapping approach in which mutant or altered forms of the mammalian PTP are used to bind (trap) one or more substrates of the PTP. Binding of the substrate trapping PTP with a substrate of the PTP results in the formation of a complex which can be readily detected, and, if desired, measurably dissociated with a suitable agent in an FP procedure that does not require separation of bound and free substrate. The mutant forms of the PTPs have attenuated catalytic activity (lack catalytic activity or have reduced catalytic activity) relative to the wild type PTP but retain the ability to bind tyrosine phosphorylated substrate(s) of the wild type PTP.

PTP Catalytic Assay

The present invention is also directed in part to a method for identifying an agent which alters the interaction between a PTP and a tyrosine phosphorylated polypeptide which is a substrate of the PTP, based on the ability of a PTP to catalytically dephosphorylate the substrate. According to these embodiments, the subject invention method comprises contacting, in the absence and in the presence of a candidate agent, a PTP and a detectably labeled PTP substrate as provided herein, under conditions and for a time sufficient to permit dephosphorylation of the substrate by the PTP, wherein the substrate is capable of generating a fluorescence energy signal. The method further comprises exposing the PTP and the substrate to a reaction terminator molecule, thereby terminating dephosphorylation of the substrate, and comparing the fluorescence energy signal level of substrate that remains phosphorylated in the absence of the candidate agent to the level of substrate that remains phosphorylated in the presence of the candidate agent. A difference in the fluorescence energy signal level indicates the agent alters the interaction between the PTP and the substrate. In certain other embodiments, the step of comparing instead comprises comparing the fluorescence energy signal level of substrate that is dephosphorylated in the absence of the candidate agent to the level of substrate that is dephosphorylated in the presence of the candidate agent, such that a difference in the fluorescence energy signal level indicates the agent alters the interaction between the PTP and the substrate.

A reaction terminator molecule includes any natural or synthetic peptide, protein, polypeptide or polypeptide analog, including an antibody or immunoglobulin sequence-derived polypeptide or fusion protein; a nucleotide, oligonucleotide, polynucleotide or other nucleic acid molecule, derivative, analog or the like; a carbohydrate (including polysaccharides and oligosaccharides and their derivatives and analogs); a lipid or an organic compound including a small molecule as provided herein, that is capable of specifically halting or substantially inhibiting catalytic dephosphorylation of a tyrosine phosphorylated polypeptide which is a substrate of a PTP, by the PTP. A reaction terminator molecule may exert its effect through interaction with a PTP substrate and/or a PTP. A reaction terminator molecule that substantially inhibits PTP-mediated catalytic dephosphorylation of a substrate preferably inhibits greater than 80% of PTP activity under defined conditions, more preferably greater than 90%, more preferably greater than 98%, still more preferably greater than 99.9% and most preferably 100%. In preferred embodiments, the step of exposing the PTP and the substrate to a reaction terminator molecule and thereby terminating dephosphorylation of the substrate is subsequent to the step of contacting the PTP and the substrate.

Examples of suitable reaction terminator molecules include, but are not limited to, an antibody specific for a PTP substrate, an antibody specific for a PTP catalytic domain, vanadate (e.g, sodium orthovanadate), an SH2 domain polypeptide (e.g., Eck et al., 1993 Nature 362:87; Waksman et al., 1992 Nature 358:646), an IRS1 PTB domain polypeptide (e.g. Eck et al., 1996 Cell 85:695), an shc PH domain polypeptide (e.g., Zhou et al., 1995 Nature 92:7784), an SH2-like domain identified in the N-terminal region of Cb1 (e.g., Meng et al., 1999 Nature 398:84) a yopH phosphotyrosine recognition domain polypeptide (e.g., Black et al., 1998 Mol. Microbiol. 29:1263) or any other non-antibody molecule that specifically binds to a tyrosine phosphorylated form of the detectable PTP substrate. In preferred embodiments the reaction terminator molecule is an antibody, which may include a monoclonal antibody, a polyclonal antibody, a Fab′ fragment, a F(ab′)₂ fragment, an immunoglobulin fusion protein, a single-chain antibody or the like. In certain particularly preferred embodiments of the invention, the reaction terminator molecule is a monoclonal antibody specific for phosphotyrosine, which antibody is not present at the initiation of the reaction wherein a PTP dephosphorylates a detectable tyrosine phosphorylated PTP substrate as provided herein, but which antibody is exposed to the PTP and the substrate at a point in time subsequent to the step of contacting the PTP and the substrate. In other preferred embodiments, the reaction terminator molecule may be an antibody specific for the detectable substrate, a substrate trapping mutant protein tyrosine phosphatase that binds to a tyrosine phosphorylated form of the detectable substrate or another molecule that specifically binds to a tyrosine phosphorylated form of the detectable substrate. For instance, a CS substrate trapping mutant PTP may be used as a reaction terminator, as described in greater detail below (see, e.g., Example 6). As another example, a suitable reaction terminator according to the present disclosure may include a molecule that specifically binds to a tyrosine phosphorylated form of the detectable substrate and may include an SH2 domain polypeptide or a PTP-PID domain polypeptide.

In certain embodiments of the present invention, a detection reagent may be further introduced into a PTP catalytic assay as described herein, wherein the detection reagent comprises any molecule capable of specifically engaging in complex formation with a detectably labeled, dephosphorylated PTP substrate and wherein the detection reagent is not a reaction terminator molecule. According to such an embodiment, such a detection reagent as well as the reaction terminator molecule are present when the step of comparing fluorescence energy signal levels is performed. Introduction of a reaction terminator molecule (e.g., vanadate) at one or more time points during such a catalytic reaction thus permits halting the dephosphorylation reaction and determination of the amount of dephosphorylated substrate. Thus, for example, continuous real-time monitoring of the degree of dephosphorylation of a substrate may be achieved where a detection reagent but not a reaction terminator molecule is present. As another example, simultaneous detection of the phosphorylation states of multiple detectably labeled, phosphotyrosine peptide PTP substrates may be achieved in a “multiplexed” embodiment of the invention wherein at least one substrate engages in specific complex formation with a reaction terminator molecule as provided herein, while at least one other distinct substrate engages in specific complex formation with a detection reagent as provided herein. Those familiar with the art will appreciate that variations and modifications of such embodiments, which employ a detection reagent and a reaction terminator molecule, are within the scope and spirit of the present invention.

As a specific example by way of illustration and not limitation, the monoclonal antibody 20G3 (nano Tools GmbH, Teningen, Germany) specifically detects the unphosphorylated polypeptide sequence that includes the tyrosine residue at amino acid position 1173, which is a site for autophosphorylation in the ligand-stimulated EGF receptor. Accordingly, fluorescently-labeled phosphopeptides derived from amino acids 1170-1176 of the EGF receptor sequence 1170-1176 (N-A-E-pY-L-R-V, SEQ ID NO: ) may be substrates for catalytically active PTPs. Furthermore, according to this example, such detectably labeled peptide substrates, upon catalytic dephosphorylation by PTP, would detectably bind to antibody 20G3 and show an increased FP signal commensurate with the degree of dephosphorylation. Termination of dephosphorylation by introduction of a reaction terminator molecule, at an appropriate time that can be determined readily and without undue experimentation based upon the teachings provided herein, then provides a source of fluorescence energy signals for comparison as described herein.

The detection of a fluorescence energy signal, according to aspects of the invention that are directed to determining levels of PTP substrate phosphorylation or dephosphorylation, includes fluorescence energy signal level determination as discussed herein. In preferred embodiments, fluorescence energy signal detection relates to FP determination as described in detail above. In other preferred embodiments, fluorescence energy signal detection as provided herein relates to determination of FRET levels wherein the detectably labeled tyrosine phosphorylated peptide substrate and the reaction terminator molecule comprise an energy transfer molecule donor-acceptor pair. Thus, in certain embodiments the substrate comprises an energy transfer acceptor molecule and the reaction terminator molecule comprises an energy transfer donor molecule. In certain other embodiments, the substrate comprises an energy transfer donor molecule and the reaction terminator molecule comprises an energy transfer acceptor molecule.

Screening assays according to this aspect of the invention as it relates to methods involving the determination of PTP substrate dephosphorylation are directed in part to comparison of (a) PTP-mediated substrate dephosphorylation in the absence of a candidate agent to (b) PTP-mediated substrate dephosphorylation in the presence of a candidate agent. For example, an agent that alters the phosphorylation state of a tyrosine phosphorylated substrate of a PTP can be identified by the methods described herein. An enzymatic activity assay utilizing the wildtype PTP may be carried out in the presence of an agent to be tested, and the resulting amount of enzyme activity (e.g., as evidenced by the level of substrate which remains phosphorylated, or, alternatively, as evidenced by the level of substrate that is dephosphorylated) can be compared with the amount of enzyme activity in the absence of the agent to be tested. A decrease in the enzymatic activity in the presence of the agent to be tested indicates that the agent inhibits the interaction between the PTP and its substrate. Conversely, an increase in the enzymatic activity in the presence of the agent to be tested indicates that the agent enhances the interaction between the PTP and its substrate. Identification and selection of suitable PTP substrates for use in these and related embodiments are as described above. The fluorescence energy signal-based detection according to this aspect of the invention may also be adapted to high throughput screening formats.

Thus, according to certain embodiments of the subject invention method, at least one PTP as provided herein is combined with at least one detectably labeled tyrosine phosphorylated polypeptide which is a substrate of the PTP, as disclosed above. Suitable assay conditions for PTP-mediated catalytic dephosphophorylation of a PTP substrate tyrosine phoshphorylated polypeptide can be readily determined without undue experimentation by a person having ordinary skill in the art, based on the disclosure herein and known properties of PTPs. Enzymatic activity assays are known in the art and may be modified according to the teachings herein; for example, assays of PTP activity using a tyrosine phosphorylated ³²P-labeled substrate are described in Flint et al. (1993 EMBO J. 12:1937-1946). For instance, a substrate may be dephosphorylated in vitro by incubating a PTP with a detectably labeled substrate peptide in a suitable buffer (e.g., Tris, pH 7.5, 1 mM EDTA, 1 mM dithiothreitol, 1 mg/mL bovine serum albumin) for 10 minutes at 30° C. In general, and depending upon the particular assay type selected (e.g., with regard to sensitivity and detection limits that may vary as a function of the reporter signal that is monitored, and further with regard to assay formats such as conventional test tubes or high throughput formats such as 96-well, 384-well or other high throughput microplates) the amounts of the reaction components may range from about 0.5-10 pg to about 50-500 ng of PTP polypeptide and from about 0.5 ng (0.1 ng for FP assays) to about 10 μg of substrate polypeptide. The extent of substrate dephosphorylation may generally be monitored by determining a fluorescence energy signal as described herein, for example, using fluorescence polarization or FRET. As noted above, a decrease in the enzymatic activity in the presence of the agent to be tested indicates that the agent inhibits the interaction between the PTP and its substrate. Conversely, an increase in the enzymatic activity in the presence of the agent to be tested indicates that the agent enhances the interaction between the PTP and its substrate.

It is contemplated that the present invention will be of major value in high throughput screening; i.e., in automated screening of a large number of candidate compounds for activity against one or more PTPs. It has particular value, for example, in screening synthetic or natural product libraries for compounds that exhibit activity in PTP binding and PTP catalytic assays as described herein. The methods of the present invention are therefore amenable to automated, cost-effective high throughput drug screening and have immediate application in a broad range of pharmaceutical drug development programs. In a preferred embodiment of the invention, the compounds to be screened are organized in a high throughput screening format such as a 96-well plate format, or other regular two dimensional array, such as a 384-well, 48-well or 24-well plate format or an array of test tubes. For high throughput screening the format is therefore preferably amenable to automation. It is preferred, for example, that an automated apparatus for use according to high throughput screening embodiments of the present invention is under the control of a computer or other programmable controller. The controller can continuously monitor the results of each step of the process, and can automatically alter the testing paradigm in response to those results.

Agents

As noted above, the invention is directed in part to a method for identifying an agent that alters PTP-substrate interactions, by combining a candidate agent with a PTP and evaluating the effect of the candidate agent on the phosphatase activity using, for example, an assay described herein. An increase or decrease in phosphatase activity can be measured by performing a representative assay according to the present disclosure, in the presence and absence of a candidate agent. Briefly, a candidate agent may be included in a mixture of active PTP polypeptide and substrate as provided herein, with or without pre-incubation with one or more components of the mixture. In general, a suitable amount of candidate agent for use in such an assay ranges from about 0.001 μM to about 100 μM. The effect of the agent on PTP activity may then be evaluated by quantifying the loss of phosphate from the substrate and comparing the loss with that achieved using the PTP in the absence of the candidate agent.

The present invention therefore also relates to assays for identifying agents which alter, e.g., enhance or inhibit, the interaction between a PTP and its phosphorylated substrate. Agents identified by these assays can be agonists (e.g., agents which enhance or increase the activity of the PTP) or antagonists (e.g., agents which inhibit or decrease the activity of the PTP) of PTP activity. The agent may be an endogenous physiological substance or may be a natural or synthetic drug, including small organic molecules.

Candidate agents for use in screening assays according to the present invention may be provided as “libraries” or collections of compounds, compositions or molecules. Such molecules typically include compounds known in the art as “small molecules” and having molecular weights less than 10⁵ daltons, preferably less than 10⁴ daltons and still more preferably less than 10³ daltons. For example, members of a library of test compounds can be administered to a plurality of samples in a high throughput screening array as provided herein, each containing at least one catalytically active PTP as provided herein, and then assayed for their ability to enhance or inhibit PTP-mediated dephosphorylation of, or binding to, a detectably labeled substrate. Compounds so identified as capable of influencing PTP function (e.g., phosphotyrosine dephosphorylation) are valuable for therapeutic and/or diagnostic purposes, since they permit treatment and/or detection of diseases associated with PTP activity. Such compounds are also valuable in research directed to molecular signaling mechanisms that involve PTPs, and to refinements in the discovery and development of future PTP-active compounds exhibiting greater specificity.

Candidate agents further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694, PCT/US91/04666, which are hereby incorporated by reference in their entireties) or other compositions that may include small molecules as provided herein (see e.g., PCT/US94/08542, EP 0774464. U.S. Pat. No. 5,798,035, U.S. Pat. No. 5,789,172. U.S. Pat. No. 5,751,629, which are hereby incorporated by reference in their entireties). Those having ordinary skill in the art will appreciate that a diverse assortment of such libraries may be prepared according to established procedures, and tested using PTPs and appropriate PTP substrates, according to the present disclosure.

Pharmaceutical Compositions/Therapeutic Methods

One or more agents which alter the interaction between at least one PTP and a tyrosine phosphorylated polypeptide which is a substrate of the PTP may also be used to alter, modulate or otherwise regulate PTP activity in a patient. As used herein, a “patient” may be any mammal, including a human, and may be afflicted with a condition associated with one or more particular PTP activities or may be free of detectable disease.

Accordingly, the treatment may be of an existing disease or may be prophylactic. Conditions associated with one or more particular PTP acitivities include any disorder associated with cell proliferation, including Duchenne muscular dystrophy, cancer, graft-versus-host disease (GVHD), autoimmune diseases, allergy or other conditions in which immunosuppression may be involved, metabolic diseases, abnormal cell growth or proliferation and cell cycle abnormalities. Certain such disorders involve loss of normal PTP activity, including activity of one or more certain MAP-kinase phosphatases, leading to uncontrolled cell growth.

For administration to a patient, one or more agents are generally formulated as a pharmaceutical composition. A pharmaceutical composition may be a sterile aqueous or non-aqueous solution, suspension or emulsion, which additionally comprises a physiologically acceptable carrier (i.e., a non-toxic material that does not interfere with the activity of the active ingredient). Such compositions may be in the form of a solid, liquid or gas (aerosol). Alternatively, compositions of the present invention may be formulated as a lyophilizate or compounds may be encapsulated within liposomes using well known technology. Pharmaceutical compositions within the scope of the present invention may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents and/or preservatives.

Any suitable carrier known to those of ordinary skill in the art may be employed in the pharmaceutical compositions of the present invention. Carriers for therapeutic use are well known, and are described, for example, in Remingtons Pharmaceutical Sciences, Mack Publishing Co. (A. R. Gennaro ed. 1985). In general, the type of carrier is selected based on the mode of administration. Pharmaceutical compositions may be formulated for any appropriate manner of administration, including, for example, topical, oral, nasal, intrathecal, rectal, vaginal, sublingual or parenteral administration, including subcutaneous, intravenous, intramuscular, intrasternal, intracavernous, intrameatal or intraurethral injection or infusion. For parenteral administration, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

A pharmaceutical composition (e.g., for oral administration or delivery by injection) may be in the form of a liquid (e.g., an elixir, syrup, solution, emulsion or suspension). A liquid pharmaceutical composition may include, for example, one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile.

The compositions described herein may be formulated for sustained release (i.e. a formulation such as a capsule or sponge that effects a slow release of compound following administration). Such compositions may generally be prepared using well known technology and administered by, for example, oral, rectal or subcutaneous implantation, or by implantation at the desired target site. Sustained-release formulations may contain an agent dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Within a pharmaceutical composition, an agent which alters PTP-substrate interactions identified according to the subject invention method may be linked to any of a variety of compounds. For example, such an agent may be linked to a targeting moiety (e.g., a monoclonal or polyclonal antibody, a protein or a liposome) that facilitates the delivery of the agent to the target site. As used herein, a “targeting moiety” may be any substance (such as a compound or cell) that, when linked to an agent enhances the transport of the agent to a target cell or tissue, thereby increasing the local concentration of the agent. Targeting moieties include antibodies or fragments thereof, receptors, ligands and other molecules that bind to cells of, or in the vicinity of, the target tissue. An antibody targeting agent may be an intact (whole) molecule, a fragment thereof, or a functional equivalent thereof. Examples of antibody fragments are F(ab′)2, −Fab′, Fab and F[v] fragments, which may be produced by conventional methods or by genetic or protein engineering. Linkage is generally covalent and may be achieved by, for example, direct condensation or other reactions, or by way of bi- or multi-functional linkers. Targeting moieties may be selected based on the cell(s) or tissue(s) at which the agent is expected to exert a therapeutic benefit.

Pharmaceutical compositions may be administered in a manner appropriate to the disease to be treated (or prevented). An appropriate dosage and a suitable duration and frequency of administration will be determined by such factors as the condition of the patient, the type and severity of the patient's disease, the particular form of the active ingredient and the method of administration. In general, an appropriate dosage and treatment regimen provides the agent(s) in an amount sufficient to provide therapeutic and/or prophylactic benefit (e.g., an improved clinical outcome, such as more frequent complete or partial remissions, or longer disease-free and/or overall survival). For prophylactic use, a dose should be sufficient to prevent, delay the onset of or diminish the severity of a disease associated with cell proliferation.

Optimal dosages may generally be determined using experimental models and/or clinical trials. In general, the amount of active agent present in a dose ranges from about 0.001 μg to about 100 μg per kg of host, typically from about 0.1 μg to about 10 μg. The use of the minimum dosage that is sufficient to provide effective therapy is usually preferred. Patients may generally be monitored for therapeutic or prophylactic effectiveness using assays suitable for the condition being treated or prevented, which will be familiar to those of ordinary skill in the art. Suitable dose sizes will vary with the size of the patient, but will typically range from about 10 mL to about 500 mL for 10-60 kg animal.

The following Examples are offered by way of illustration and not by way of limitation.

### EXAMPLES

### Example 1

### Generation, Expression and Purification of Mutant PTP Proteins

Plasmid isolation, production of competent cells, transformation and related manipulations for the cloning, amplification, construction of recombinant plasmids, inserts and vectors, sequencing and the like, were carried out according to published procedures (Sambrook et al., Molecular Cloning. a Laboratory Manual, Cold Spring Harbor Laboratory Press, Cold Spring Harbor, N.Y., 1989; Ausubel et al., 1993 Current Protocols in Molecular Biology, Greene Publ. Assoc. Inc. & John Wiley & Sons, Inc., Boston, Mass.). Recombinant nucleic acid expression constructs encoding human PTP-PEST (Garton et al., 1994 EMBO J. 13:3763; Garton et al. 1996 Mol. Cell. Biol. 16:6408) and human PTP-1B (Brown-Shimer et al., 1990 Proc. Nat. Acad. Sci. USA 87:5148) were prepared as described.

Point mutations within the catalytic domains of PTPs were introduced using standard procedures, for example, the invariant aspartate (D) at amino acid position 199 in PTP-PEST being converted to alanine (A) by a substitution mutation (D199A). Thus, mutations giving rise to PTP-PEST(D199A), PTP-PEST(C231S), PTP1B(D181A) and PTP1B(C215S) were introduced by site-directed mutagenesis using the Muta-Gene™ in vitro mutagenesis kit (Bio-Rad, Richmond, Calif.) according to the manufacturer's instructions. Regions containing the specified point mutation were then exchanged with the corresponding wild type sequences within appropriate expression vectors, and the replaced mutant regions were sequenced in their entirety to verify the absence of additional mutations.

PTP1B proteins (wild type and mutant forms) comprising amino acids 1-321 were expressed in E. coli and purified to homogeneity as described in Barford et al. (J. Mol. Biol. 239:726-730 (1994)).

### Example 2

### Synthesis of Fluorescently-labeled Phosphotyrosyl-containing Substrate Peptides

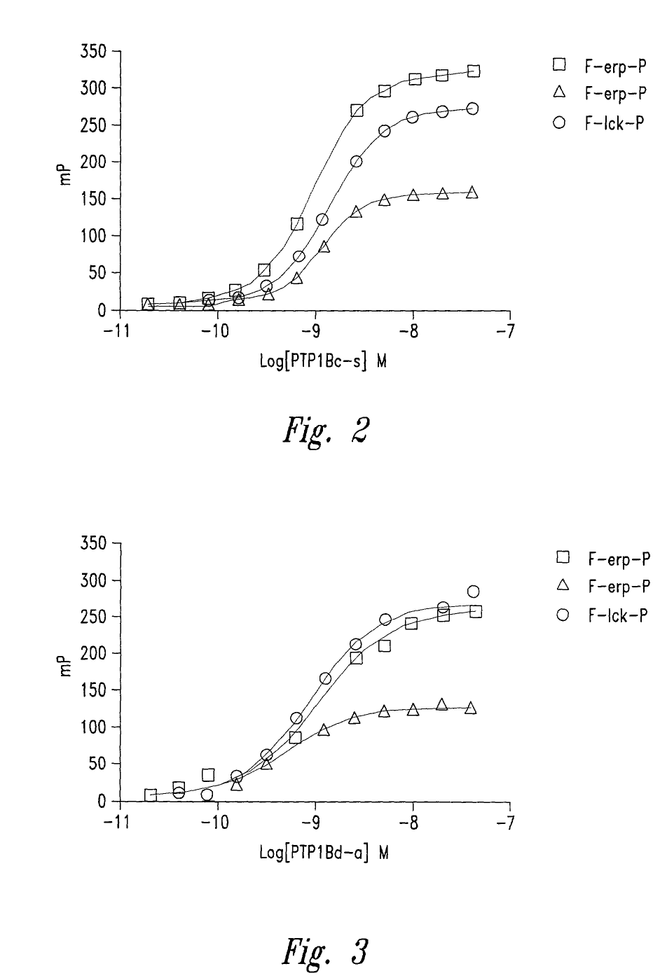

For assays of PTP1B many proteins and peptides function as substrates with preferred substrates containing acidic amino acid residues on the N-terminal side of the phosphotyrosine. One of the preferred substrates for PTP I B is derived from autophosphorylation site tyrosine 992 of the EGF receptor. The sequences surrounding this phosphorylation site can be reduced to a hexapeptide without compromising the favorable kinetic properties of this substrate, i e Km of ˜3 uM with a kcat of 72 s⁻¹ (Zhang et al., 1994 Biochemistry 33:2285-2290). The molecular basis for the recognition of this substrate by PTP1B has been determined by X-ray crystallographic determination of the structure of this peptide (and truncated versions of it) bound to the catalytically inactive C215S mutant of PTP1B (Jia et al., 1995 Science 268:1754-1758). The sequence of this peptide, F-erp-P (FIGS. 2, 3) D-A-D-E-pY-L-NH₂ (SEQ ID NO: corresponds to residues 988-993 of the human EGF receptor. This peptide is available commercially (Bachem Bioscience Inc., King of Prussia, Pa.) and can be readily synthesized according to established methodologies.

The fluorescein-labeled form of the peptide was made by coupling 5′carboxyfluorescein to the amino terminus of the peptide using a succinimidyl ester activated form of fluorescein (single isomer 5′-carboxyfluorescein succinimidyl ester, Molecular Probes, Eugene, O R, cat # C2210). The reaction conditions were essentially as recommended by the manufacturer, 4 mM phosphopeptide, 10 mM 5′-carboxyfluorescein succinimidyl ester in 300 mM sodium bicarbonate buffer for 2 hrs at room temperature. Excess reagent was quenched with 60 mM hydroxylamine. Fluorescein-peptide was purified by reverse phase HPLC and the structure was confirmed by mass spectrometry. The concentration of fluorescein-labeled phosphopeptide was calculated using absorbance of fluorescein with an extinction coefficient of 73,500 cm⁻¹M⁻¹.

For comparison to the EGF-receptor derived peptide two other phosphotyrosyl containing substrates were prepared. One, F-IR-P (FIGS. 2, 3), which was based on residues 1142-1152 of the human insulin receptor, encompassed three autophosphorylation sites tyrosine 1146, 1151 and 1152. A peptide with only tyrosine 1146 phosphorylated was synthesized at Princeton Biomolecules (Columbus, Ohio) and labeled with 5′-carboxyfluorescein on the N-terminus, and purified by HPLC. This peptide with sequence T-R-D-I-pY-E-T-D-Y-Y-R (SEQ ID NO: ) is one of the best reported substrates for PTPs LAR and CD45 with Km's of 27 uM and 34 uM respectively (Cho et al., 1992 Biochemistry 31:133-138).

The third peptide utilized in this example, F-lck-P (FIGS. 2, 3 ) corresponded to residues 500-509 of p56lck, the src-like lymphocyte specific protein tyrosine kinase that is a physiological substrate for CD45. The peptide chosen represents the inhibitory phosphorylation site in the C-terminal regulatory segment of p56lck and has a reported Km of 130 uM toward CD45 (Cho, H., et al., 1992 Biochemistry 31:133-138). Its sequence was A-T-E-G-Q-pY-Q-P-Q-P (SEQ ID NO: ). This substrate peptide was synthesized, labeled with fluorescein and purified by HPLC by SynPep Corporation, Dublin Calif.

### Example 3

### Characterization of Phosphotyrosyl Substrate Binding by C215S Mutant and D181A Mutant of PTP1B

To characterize the binding interaction between three different substrate peptides described in Example 2, F-erp-P, F-IR-P and F-lck-P, and substrate binding mutants of PTP1B in solution (described in Example 1), the change in fluorescence polarization of the fluorescein labeled-substrate was measured in the presence of different concentrations of the mutant PTP. Two assay parameters were measured: (1) the dynamic range of the signal (i.e. the difference between polarization values of the bound and the unbound fluorescent substrate) and (2) the midpoint of the transition between bound and unbound that represents the apparent Kd (i.e. affinity) of each binding mutant for each peptide.

Substrate peptides and binding protein (CS or DA mutant of PTP1B) were diluted in freshly prepared Assay Buffer comprised of 25 mM Tris HCl pH 7.5, 1 mM EDTA, 2 mM DTT and 0.1 mg/ml bovine gamma globulin. Assays were set up at room temperature in black polypropylene 96 well microtiter plates (Polyfiltronics, Whatman, U.K.) by the sequential addition of 40 ul of Assay Buffer, 40 ul of diluted binding protein and 40 ul of 0.3 nM fluorescein-labeled substrate peptide. After 15 min plates were read in a Criterion Analyst™ (LJL BioSystems, Inc., Sunnyvale, Calif.) in fluorescence polarization detection mode using the parameters listed as follows:

| | | |
|---|---|---|
| Microplate | Polyfiltronics PP96 | |
| format: | | |
| Detection mode: | FP | |
| Units: | cps | |
| Lamp: | Continuous | |
| Excitation side: | Top | |
| Excitation filter: | 1 | Fluorescein 485 nm |
| Excitation | s | |
| polarizer filter: | | |
| Photon | counting HC-120 | |
| head: | | |
| Emission side: | Top | |
| Attenuator mode: | o | |
| Emission filter: | 1 | Fluorescein 530 nm |
| Emission | s | |
| polarizer filter: | | |
| Z Height: | 1 | mm |
| Conversion | Comparator | |
| method: | | |
| A/D | converter x1 | |
| gain | | |
| Integrating gain | x1 | |
| Integrating | 0 | |
| capacitor | | |
| Read sequence: | row | |
| Integration Time: | 500000 | us |
| Total integration | 500000 | us |
| time: | | |
| Readings | per 1 | |
| well: | | |
| Time | between 100 | ms |
| readings: | | |
| Delay after flash: | 0 | us |
| G factor: | 1 | |
| Dynamic | Emission | Polarizer |
| polarizer: | | |
| Polarizer settling | 30 | ms |
| time: | | |
| Shake Time: | 0 | s |
| Temperature: | 26.6 | C |
| Instrument tag: | Set | by |
| | customer | |
| Serial number: | AN0088 | |
| Well List: | A1:H12 | |
| Data: | Polarization | |
| Units: | mP | |

To obtain values for background fluorescence, assays were set up as above except the fluorescein-labeled substrate was replaced with Assay Buffer. To determine the fluorescence polarization values for the unbound substrate peptide, assays were set up as above except that the 40 ul of diluted binding protein was replaced with Assay Buffer. Data were recorded for each of the 96 wells of a plate as fluorescence detected in the S channel for light emitted in the same plane as the exciting light and in the P channel for light emitted in the plane perpendicular to that of the exciting light. The fluorescence polarization values, reported in mP units, were calculated from the fluorescence measured in the S and P channels. To calculate mP, the background fluorescence measured in the S channel was subtracted from each measurement of fluorescence in the S channel and the background fluorescence measured in the P channel was subtracted from each measurement of fluorescence in the P channel. Then using these corrected values, mP=1000*(S−P)/(S+P). A G factor of 1 was used for these calculations and thus does not show up in this equation. This factor is required under certain circumstances to adjust the fluorescence values in the P channel. Additional corrections as described in an FP-based protease assay (Levine et al., 1997 Anal. Biochem. 247:83-88) also were unnecessary as no quenching of the fluorophore was observed upon its being bound.

The results of the binding assays with the CS binding mutant of PTP1B (FIG. 2 and Table 1) indicated that the EGF-receptor derived peptide (F-ERP-P) gave the largest dynamic range with a maximum polarization value of 315 mP for the fully bound peptide substrate. The substrate corresponding to the C-terminal segment of p56lck had the next largest polarization value for the bound peptide of 266 mP, whereas the insulin receptor derived peptide (F-IR-P) obtained a maximum polarization value of 151 mP. The apparent Kd's for each of these peptides were very similar, ranging in value between 0.97 nM and 1.4 nM. Based on the larger dynamic range achieved with the EGF receptor derived sequence, this substrate peptide was more commonly used in later assays.

TABLE 1
PTP-1B (CS) Binding Assay

| Variable | F-ERP-P | F-IR-P | F-lck-P |
|---|---|---|---|
| Peak mP | 315.6 | 151.4 | 266.4 |
| EC50 | 9.650e-010 | 1.160e-009 | 1.43e-009 |

The results obtained with the DA binding mutant of PTP1B (FIG. 3 and Table 2) were overall quite similar to those measured with the CS mutant, although the maximum polarization achieved upon binding the EGF-receptor derived peptide was somewhat lower (269 mP). The maximum polarization values for bound insulin receptor peptide was 132 mP and for the p56lck peptide was 277 mP. The Kd's ranged between 0.5 nM and 1.1 nM.

TABLE 2
PTP-1B (DA) Binding Assay

| Variable | F-ERP-P | F-IR-P | F-lck-P |
|---|---|---|---|
| Peak mP | 269.0 | 132.3 | 276.9 |
| EC50 | 1.072e-009 | 5.079e-010 | 9.099e-010 |

### Example 4

### Measurement of Inhibition of Peptide Binding by Various Agents

Binding assays were set up as described in Example 3, except that the first 40 ul addition to the black polypropylene microtiter plate was not Assay Buffer but instead was a dilution of any test agents, compounds, or inhibitors in buffer comprised of 25 mM Tris HCl pH 7.5, 3% DMSO. To the test agents was added 40 ul of 3 nM CS or DA binding mutant of PTP1B in Assay Buffer. Plates were shaken for 30 sec to mix the two solutions. After another 12.5 minutes 40 ul of 3 nM fluorescein-labeled EGF receptor peptide was added and the solutions mixed for 30 sec. After 12.5 minutes of incubation the plates were read as described in Example 3.

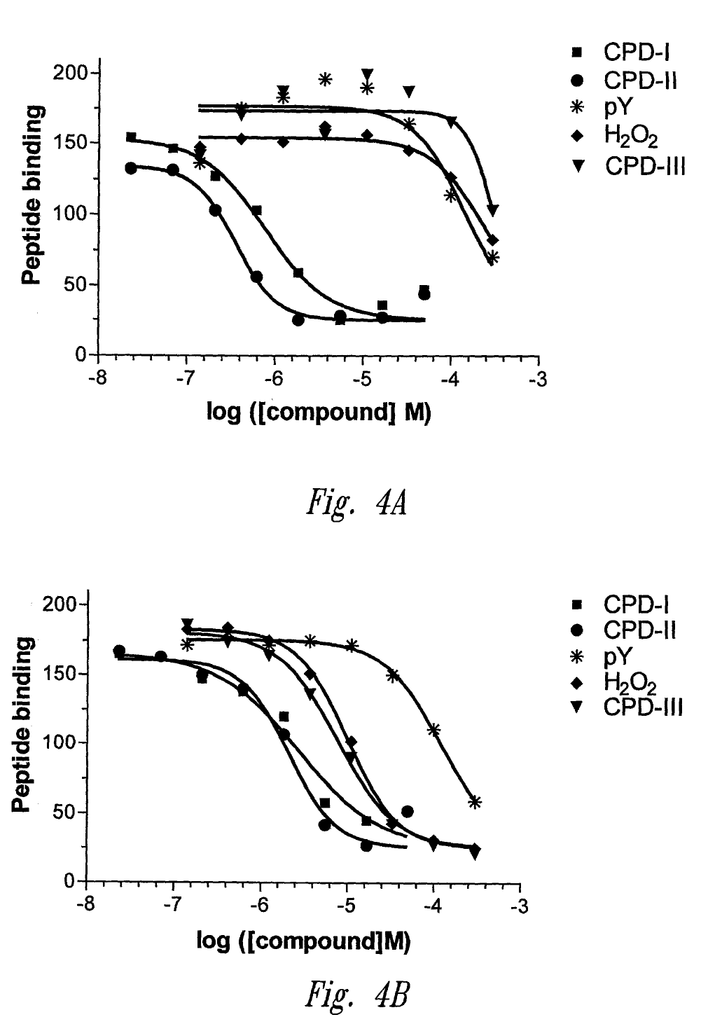

Agents such as phosphotyrosine or nonfluorescent phosphotyrosyl peptides inhibited binding of fluorescent peptide substrate to both CS (FIG. 4A) and DA (FIG. 4B) mutants of PTP1B with similar potency (FIG. 4 and Table 3). Small molecule inhibitors such as CPD-I and CPD-II also inhibited both CS and DA mutants of PTP1B, although each compound was several fold more potent toward the CS mutant than toward the DA mutant. Agents such as hydrogen peroxide and small organic molecule CPD-III inhibited the DA substrate binding mutant with an IC50 of approximately 10 uM but failed to inhibit the CS substrate binding mutant of PTP1B until their concentrations were well in excess of 100 uM. This stark difference in IC50s highlighted a major utility of the CS binding assay, which was to indicate which agents were acting to inhibit PTPs through a chemically reactive process dependent upon the catalytic cysteine residue. In this example, hydrogen peroxide inhibited PTP activity and the binding of peptide substrate by the DA binding mutant by oxidation of the cysteine thiol. Treatment with CPD-III also promoted a covalent chemical modification of the catalytic site thiol.

TABLE 3
Comparison of Substrate Binding Assays Using C215S and D181A PTP1B

| | C215S | D181A |
|---|---|---|
| Compound | IC₅₀ (μM) | IC₅₀ (μM) |
| CPD-I | 0.78 | 2.7 |
| CPD-II | 0.30 | 2.1 |
| P-Tyr | 150 | 120 |
| H₂O₂ | 250 | 10 |
| CPD-III | 810 | 7.7 |

### Example 5

### Characterization of an Assay for Measuring Dephosphorylation of Fluorescent-phosphotyrosyl Peptides with FP-based Detection

In establishing any reliable enzymatic assay, the readout, whether a change in absorbance, fluorescence, radioactivity or any other quantifiable output must be directly proportional to the amount of enzyme added to the reaction. To determine the useful operating range of the assay the limits of this linear region must be identified both in terms of quantity of enzyme utilized and in the duration of the assay. In this example PTP1B was used to dephosphorylate the fluorescein-labeled EGF receptor derived phosphopeptide substrate F-erp-P (see Example 2). The amount of phosphopeptide substrate remaining after treatment with PTP1B was assessed by forming a complex between the phosphopeptide and an antiphosphotyrosine monoclonal antibody and measuring the extent of fluorescence polarization. In addition to serving as a detection agent, the antibody used in this example, G104 (obtained from Cold Spring Harbor Laboratory, Cold Spring Harbor, N.Y.), also served as a reaction terminator by stopping any further dephosphorylation of the peptide substrate.

Initially a standard curve was established to assess the linear range of FP-based detection for determining the phosphopeptide content of an assay. To mimic conditions of a reaction, fluorescein-labeled phosphotyrosyl peptide substrate (F-ERP-P, prepared as described in Example 2) was mixed with fluorescein-labeled peptide of the same sequence but lacking phosphorylation of the tyrosine residue (F-ERP). The total amount of peptide in the assay was kept constant at 1 nM and solutions containing different ratios of phospho- and dephospho-peptide were prepared that covered the entire range from 100% phosphopeptide to 0% phosphopeptide (i.e. 100% dephosphopeptide).

As described in Example 3, assays were performed in black polypropylene 96 well plates and counted on the LJL Criterion™ Analyst. Solutions of F-ERP-P and F-ERP peptide at 3 nM each were prepared in Assay Buffer. These two solutions were mixed in 10 different proportions to create batches of fluorescent peptide that were 100%, 90%, 80%, 70%, 60%, 50%, 40%, 30%, 20%, 10% and 0% phosphorylated (e.g for 100% phosphorylated take 1 ml of F-ERP-P, for 90% phosphorylated mix 0.9 ml of F-ERP-P with 0.1 ml F-ERP, for 80% phosphorylated mix 0.8 ml of F-ERP-P with 0.2 ml of F-ERP, etc.). Five assays were set up for measuring the polarization values at each ratio of F-ERP-P/F-ERP. To each well was added 40 ul of Assay Buffer, 40 ul of F-ERP-P/F-ERP mixtures and 40 ul of antiphosphotyrosine monoclonal antibody G104 (ascites fluid diluted 1:1000 in Assay Buffer). Previous titration experiments had shown that this quantity of G104 was at least 5 fold more than was required to bind all 1 nM of F-ERP-P in the assay. A set of 36 wells was used to assess background fluorescence by setting up assays in which the 40 ul of fluorescent peptide was replaced with Assay Buffer. After 15 min, assay plates were read and calculations of mP values were made as described in Example 3.

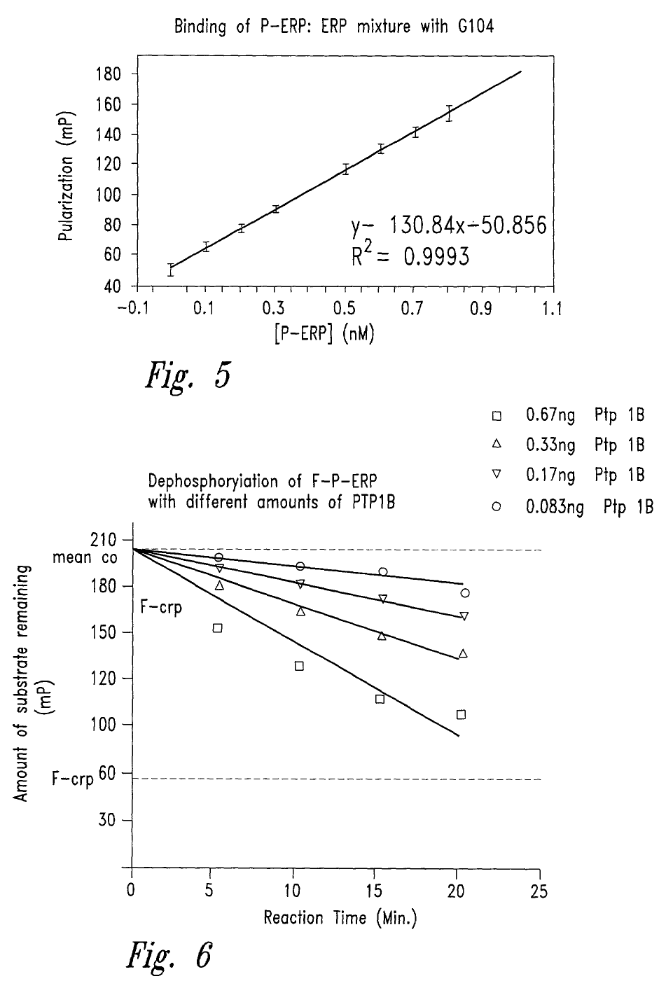

The results, shown in FIG. 5, indicated that the polarization values for this fluorescent peptide substrate in a complex with G104 were linearly proportional to the amount of phosphopeptide in the assay across the entire range from 0% to 100%. Data points represent the average value from the five determinations, and the error bars represent the standard deviation. The polarization value obtained with 100% dephosphopeptide F-ERP (i.e. 0% phosphopeptide) was the same as that obtained with fluorescent peptide in the absence of antibody G104 which indicated that under these assay conditions there was no measurable nonspecific binding of the antibody to the dephosphorylated peptide. Furthermore, the small, constant standard deviation that was measured across the range of this assay indicated that these materials and instrumentation would allow a reliable assay for PTP activity to be established.

To establish the useful linear working range of the assay for PTP catalyzed dephosphorylation of a fluorescent phosphotyrosyl peptide substrate with FP-based detection, assays were set up with four different amounts of PTP1B and were stopped at four timed intervals. To black polypropylene plates was added 20 ul of 25 mM Tris HCl pH 7.5 (or if an agent were being tested for its ability to affect the activity of the PTP, it would be added to the plates, in the same buffer but with the addition of 3% DMSO as a vehicle control) and 20 ul of Assay Buffer containing different quantities of PTP1B prepared as described in Example 1 (0.67 ng, 0.33 ng, 0.17 ng, or 0.083 ng). After mixing for 0.5 min and allowing another 12.5 min of incubation, 20 ul of 6 nM F-ERP-P in Assay Buffer was added and mixed for 0.5 min. After 5. 10, 15 or 20 minutes 60 ul of G104 (1:4000 dilution of ascites) in Assay Buffer was added to terminate the dephosphorylation reaction and form a complex with the remaining F-ERP-P. A mock reaction lacking any PTP1B provided the maximum polarization values, and a reaction in which no G104 was included provided the minimum polarization values that were equivalent to completely dephosphorylated peptide (see FIG. 5). As in Example 3, wells for making measurements of background fluorescence (i.e. in the absence of added F-ERP-P) were also included in the experiment. Fifteen minutes after the addition of G104, plates were read and mP values were calculated as described in Example 3. The PTP activity was calculated from the fraction of the total F-ERP-P substrate (i.e. change in mP values since time zero as a fraction of the total difference in mP between measured minimum and maximum polarization values) that was dephosphorylated during the time of the reaction.

The results of this experiment are shown in FIG. 6. For polarization values greater than 150 mP (equivalent to less than 30% dephosphorylation of peptide substrate) the dephosphorylation reactions were linear with time of incubation and the rates of dephosphorylation were proportional to the amount of PTP1B added to the reaction.

### Example 6

### Use of an Alternate Terminator Molecule in an Assay for Measuring Dephosphorylation of a Fluorescent-phosphotyrosyl Peptide Substrate

This example shows that following termination of the catalytic PTP assay dephosphorylation reaction, the product that is detected remains stable for many hours afterwards. In this experiment a comparison was made between terminating the assay with either of two different reaction terminator molecules, the antiphosphotyrosine monoclonal antibody G104 or the CS mutant of PTP1B. The ability of each reaction terminator molecule also was assessed in the presence and absence of vanadate (a well known nonspecific PTP inhibitor). A priori it was not known whether the phosphopeptide binding proteins that are necessary for forming the complex that results in increased polarized fluorescence of the fluorescent-phosphotyrosyl peptide substrate would also serve to completely inhibit the activity of the PTP in the assay.

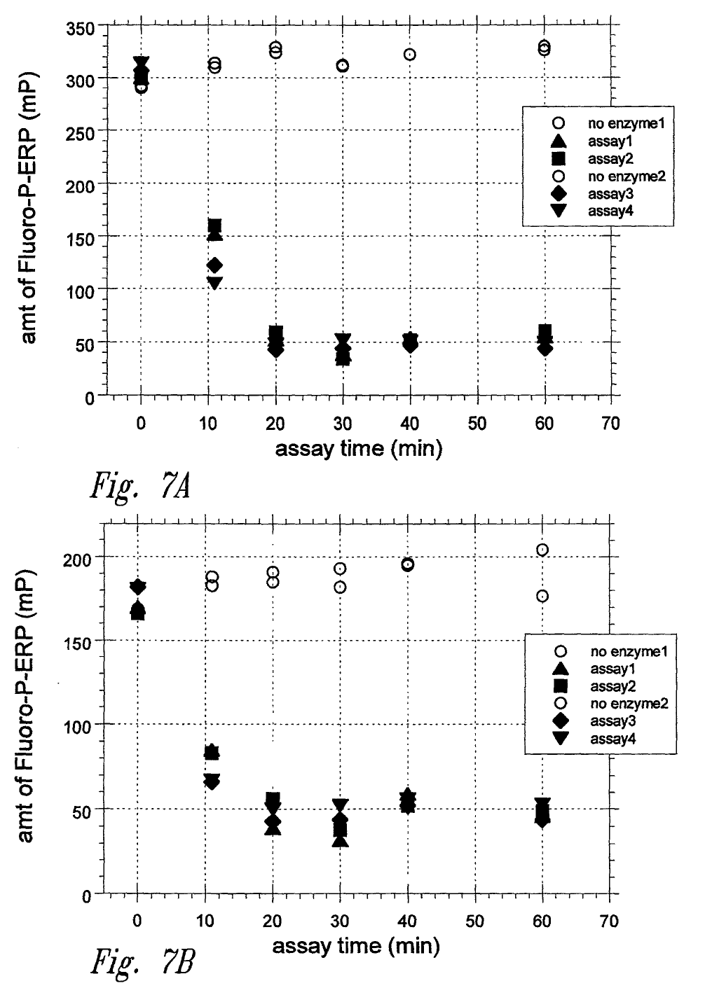

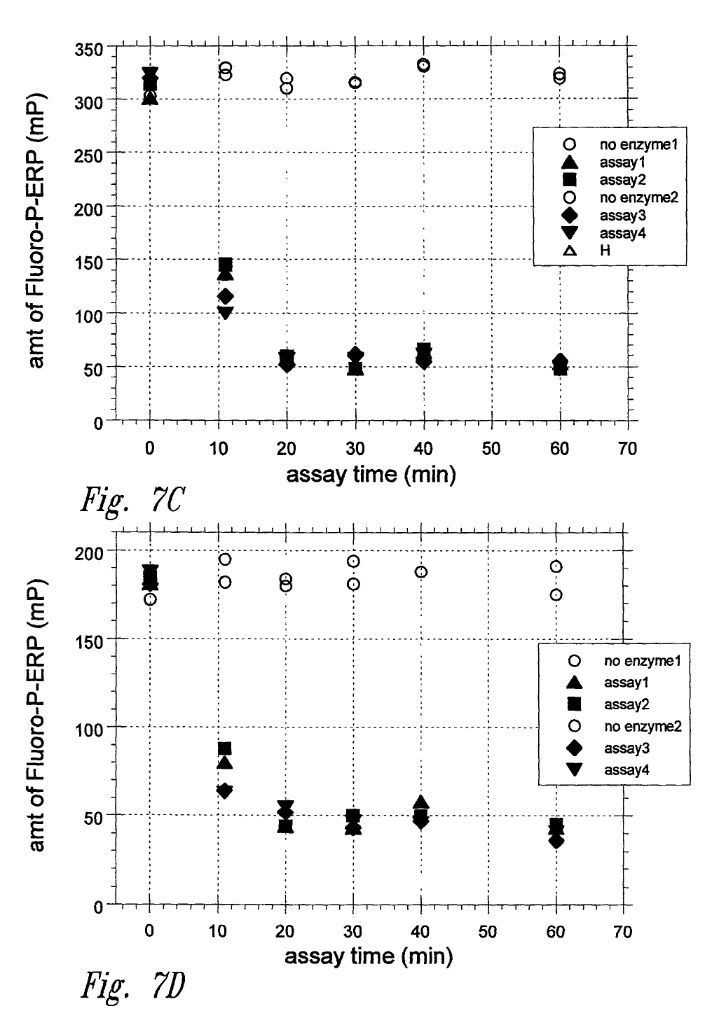

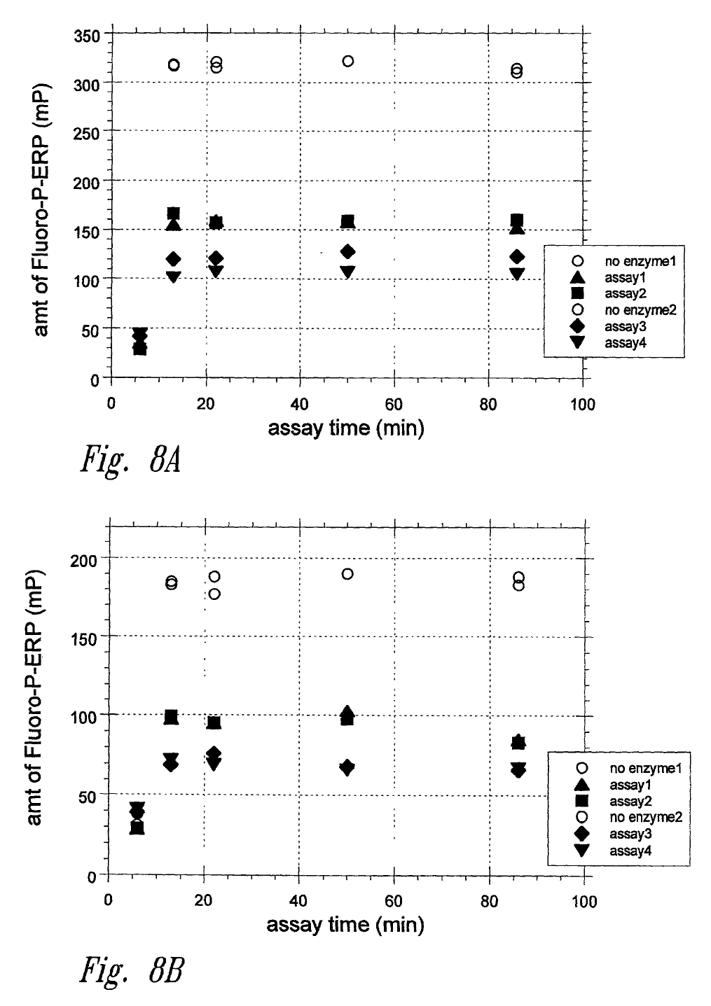

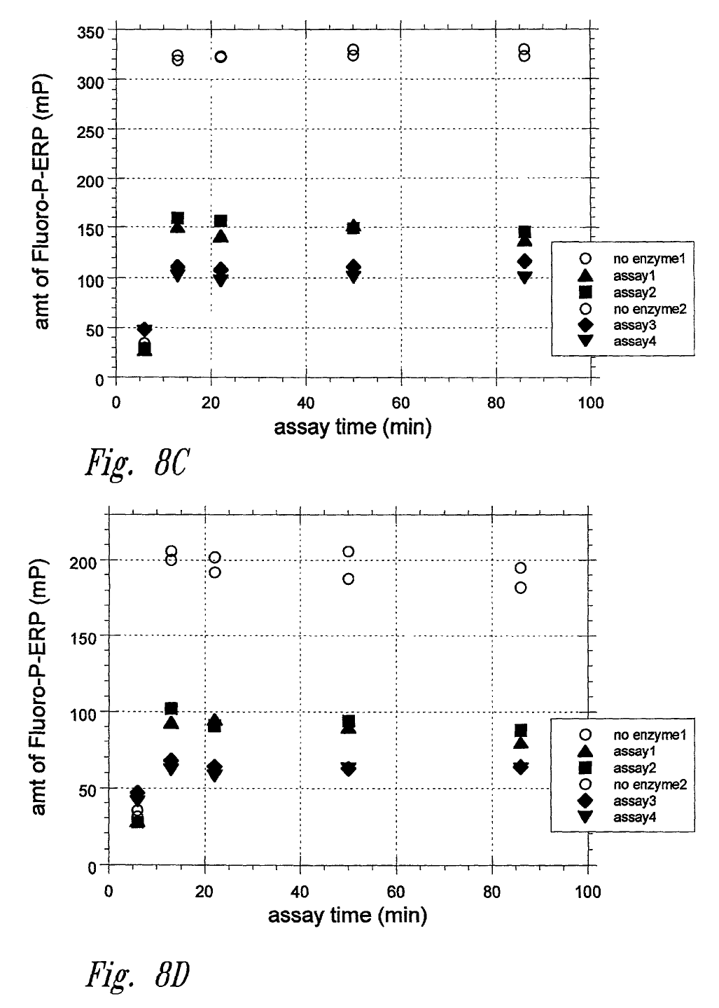

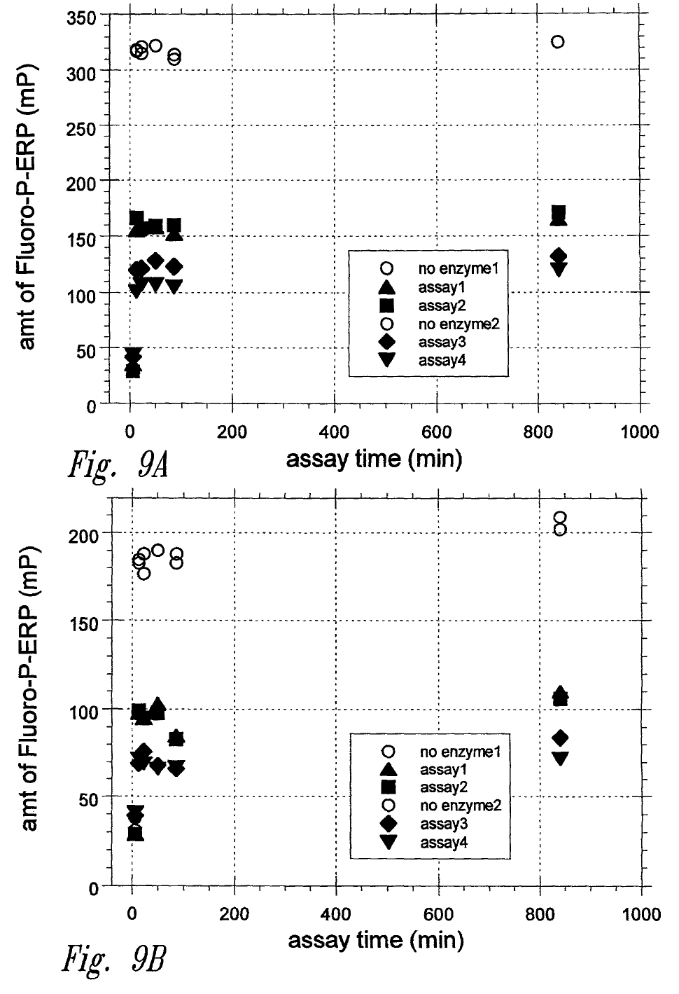

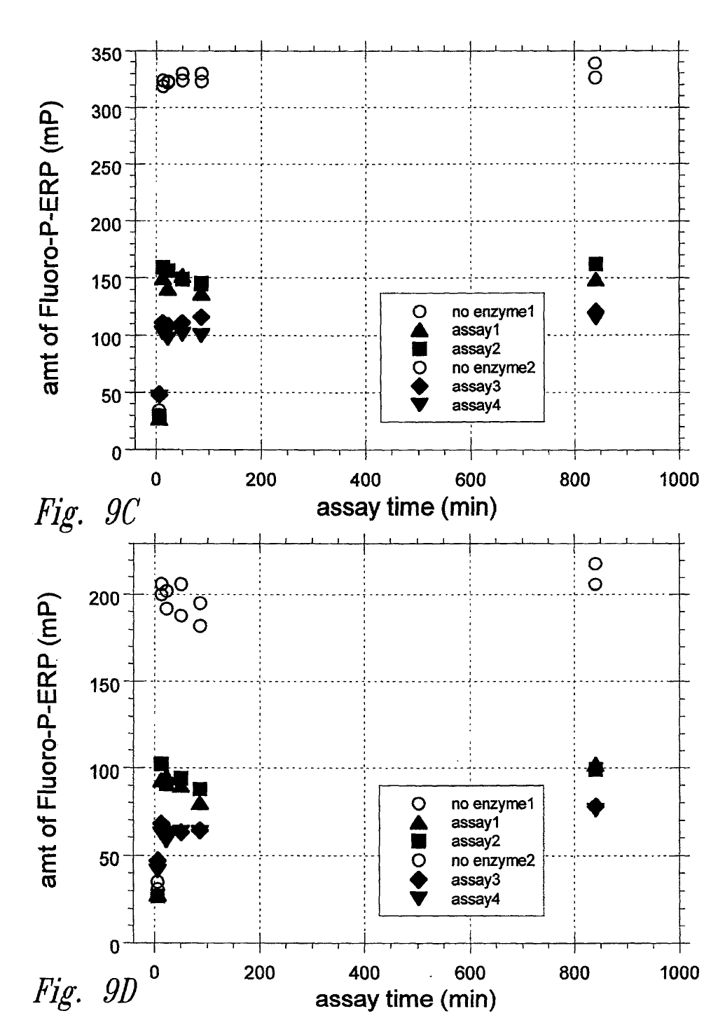

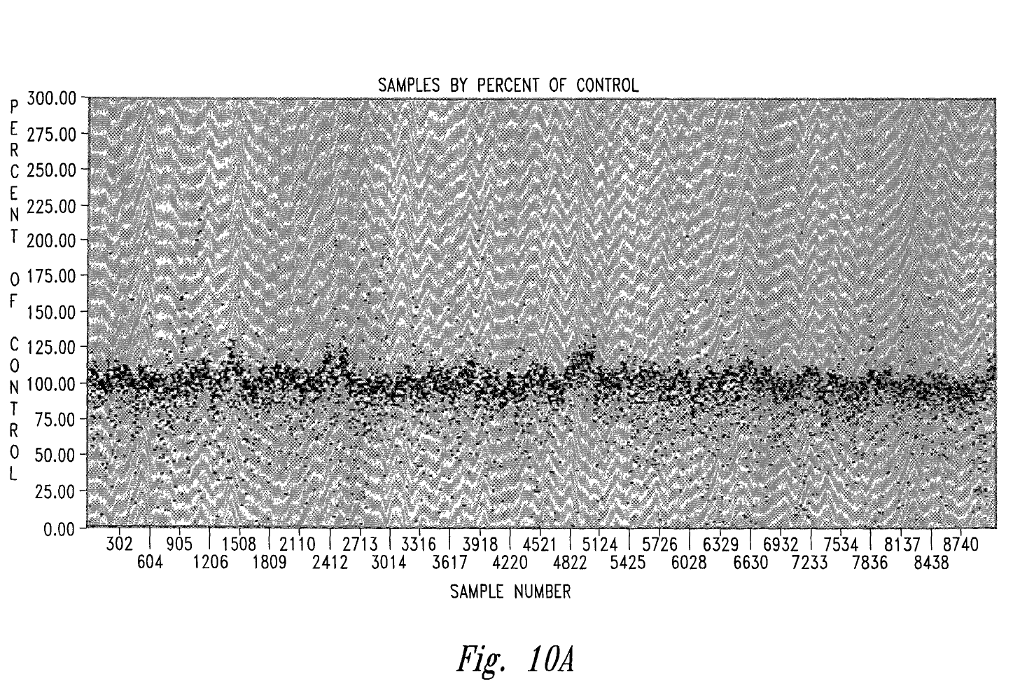

Assays were set up in black polypropylene plates with 20 ul of 25 mM Tris HCl pH 7.5 and 20 ul of 300 nM F-ERP-P in 25 mM Tris HCl pH 7.5, 1 mM EDTA, 1 mM DTT, 0.25 mg/ml ovalbumin (TEOD buffer). The dephosphorylation reaction was initiated upon addition of 20 ul of 1.5 ng/ml PTP1B in TEOD buffer. At 11 min, 20 min, 30 min. 40 min and 60 min independent sets of assays were terminated with addition of 60 ul of 4 different solutions (1) 2 uM C215S PTP1B in TEOD buffer (FIGS. 7A, 8A, 9A), or (2) 2 uM C215S PTP1B, 2 mM sodium ortho vandadate in TEOD buffer (FIGS. 7C, 8C, 9C), or (3) G104 (1:70 dilution of ascites in TEOD buffer)(FIGS. 7B, 8B, 9B), or (4) 2 mM sodium ortho vanadate, G104 (1:70 dilution of ascites in TEOD buffer (FIGS. 7D, 8D, 9D). For a zero time point, reactions received 60 ul of terminator solution prior to addition of 20 ul of 1.5 ng/ml PTP1B in TEOD buffer. To assess the course of the dephosphorylation reaction and to ascertain whether any polarization values were changing after addition of the different terminator solutions, plates were read at 6 min, 13 min, 22 min, 50 min, 86 min, and 14 hr after the dephosphorylation reaction was initiated. Plates were read using the Polarstar instrument from BMG Lab Technologies using the following settings:

| | |
|---|---|
| Gain | 067 072 |
| Excitation filter | 485 |
| Emission filter | 520 |
| Test type | plate mode |
| Reading direction | horizontal |
| No. of cycles | 1 |
| No. of flashes | 10 |
| Int. Delay (us) | 0 |
| Int. Time (us) | 12 |
| Shake time (s) | 30 before each cycle |
| Measurement type | polar |
| Start | 1 |
| Stop | 1 |
| Cycle time | 154 |
| K factor | 0.992 |

As in Example 3, wells were set up to measure background fluorescence to allow proper calculation of mP values and other wells were set up without addition of terminator reagent to assess the minimum polarization value of the unbound fluorescent phosphotyrosyl peptide (F-ERP-P).

The results are depicted in FIGS. 7-9. First, under these conditions the dephosphorylation of F-ERP-P was complete by 20 minutes. Second, monoclonal antiphosphotyrosine antibody G104 and the C215S-PTP1B binding mutant were equally effective at stopping the dephosphorylation reaction, and the polarization values for each set of reactions, which were stopped after 11 min, remained stable for 14 hours. Third, inclusion of sodium ortho vanadate in the terminator mixes did not appear to have any additive effect on the observed endpoints-both of the reaction terminator molecule proteins, when tested alone, effectively stopped the dephosphorylation reaction and created stable complexes with the remaining fluorescent phosphopeptide. Fourth, the maximum polarization values for bound peptide in this set of experiments were 300 mP for the CS mutant of PTP1B and 190 mP for G104. Thus, in these experiments, the use of the CS mutant of PTP1B as a reaction terminator molecule created a larger window of mP values in which to calculate the assay results.

### Example 7

### Use of PTP Binding and Dephosphorylation Assays of a Fluorescent-phosphotyrosyl Peptide Substrate with FP Detection in High Throughput Screening of Small Molecule Compounds

In this example, both the assay for PTP-fluorescent-phosphotyrosyl peptide substrate binding and the PTP catalytic assay for dephosphorylation of fluorescent-phosphotyrosyl peptide substrate, coupled with FP based detection, were tested in high throughput screening for detection of modulators of PTP function. PTP1B, C215S-PTP1B, F-ERP-P as substrate and a collection of 10,000 chemically diverse small organic molecules were analyzed.

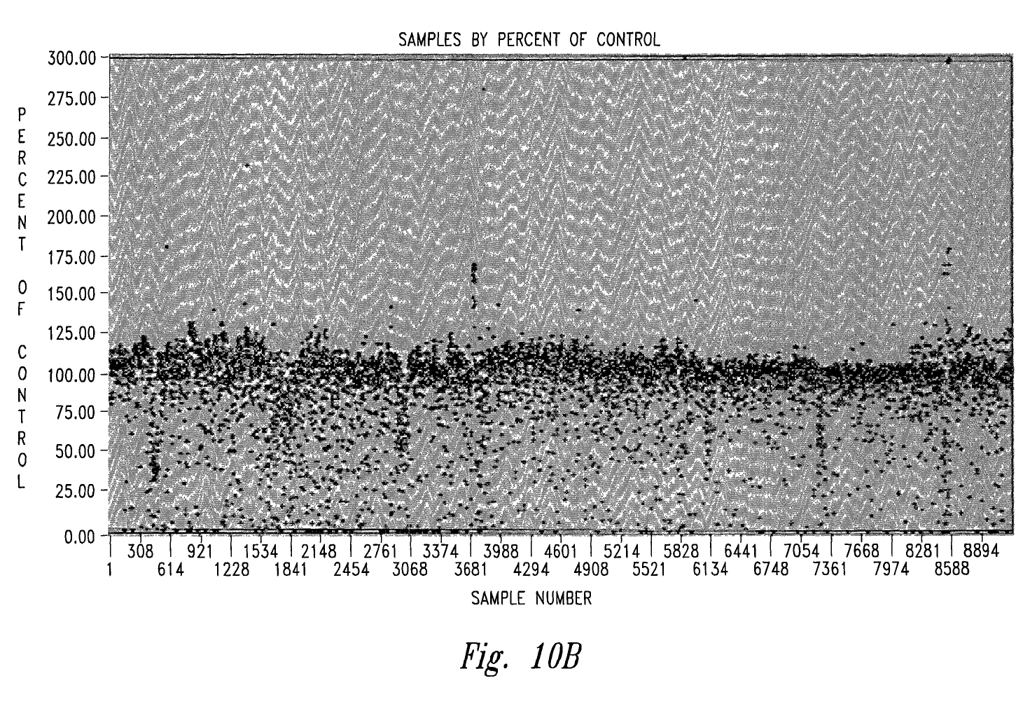

For the binding assay, assays were set up in black polypropylene plates essentially as described in Example 4. Chemical compounds (candidate agents) were dispensed into plates at 1 uM in 40 ul of 25 mM Tris HCl pH 7.5, 3% DMSO. 40 ul of 6 nM CS binding mutant of PTP1B in Assay Buffer was added and incubated for 13 min prior to addition of 40 ul of 3 nM of F-ERP-P. After 13 min incubation at room temperature, plates were read in LJL Criterion™ Analyst and calculations of mP values were performed as described in Example 3. Some wells on each plate contained no test compounds and these polarization values for uninhibited binding assays were used to normalize results between plates to create the scattergram analysis of the entire screen shown in FIG. 10A. The results in FIG. 10A show that the vast majority of compounds in this collection were without effect on the binding activity and that active compounds in this assay could be quite clearly distinguished from the inactive majority.

For the FP-based assay to measure dephosphorylation of fluorescent phosphotyrosyl peptide (F-ERP-P), assays were set up in black polypropylene plates. Chemical compounds (candidate agents) were dispensed into plates at 20 uM in 20 ul of 25 mM Tris HCl pH 7.5, 3% DMSO. 20 ul of 1 ng/ml PTP1B in Assay Buffer was added, mixed for 0.5 min and incubated for another 12.5 min at which time 20 ul of 6 nM F-ERP-P in Assay Buffer was added. The reaction was mixed for 0.5 min and allowed to proceed for another 12.5 min before being stopped by the addition of 60 ul monoclonal anti-phosphotyrosine antibody G104 as the reaction terminator molecule (ascites fluid diluted 1:4000 in Assay Buffer). As described above for the binding assay, control wells in which no test compound was added, and wells to which no enzyme were added, were used to normalize results between plates and to create the scattergram in FIG. 10B. The results presented in FIG. 10B demonstrate that this assay performed satisfactorily in a high throughput screening format such that active compounds could be clearly distinguished from a large background of compounds which did not inhibit PTP-mediated dephosphorylation of substrate.

Those skilled in the art will recognize, or be able to ascertain, using no more than routine experimentation, many equivalents to the specific embodiments of the invention described herein. Such equivalents are intended to be encompassed by the following claims:

1. A method for identifying an agent which alters the interaction between a protein tyrosine phosphatase and a tyrosine phosphorylated polypeptide which is a substrate of the protein tyrosine phosphatase, comprising:
(a) contacting in the absence and in the presence of a candidate agent, a substrate trapping mutant of a protein tyrosine phosphatase and a detectably labeled tyrosine phosphorylated peptide which is a substrate of the protein tyrosine phosphatase under conditions and for a time sufficient to permit formation of a complex between the tyrosine phosphorylated peptide and the substrate trapping mutant protein tyrosine phosphatase, wherein the substrate is capable of generating a fluorescence energy signal; and
(b) comparing the fluorescence energy signal level in the absence of the agent to the fluorescence energy signal level in the presence of the agent, wherein a difference in the fluorescence energy signal level indicates the agent alters formation of a complex between the protein tyrosine phosphatase and the substrate.

2. The method of claim 1 wherein the fluorescence energy signal is a fluorescent polarization signal.

3. The method of claim 1 wherein the detectably labeled tyrosine phosphorylated peptide comprises a fluorophore.

4. The method of claim 3 wherein the fluorophore is selected from the group consisting of fluorescein, rhodamine, Texas Red, AlexaFluor-594, AlexaFluor-488, Oregon Green, BODIPY-FL and Cy-5.

5. The method of claim 1 wherein the substrate comprises a polypeptide sequence derived from a protein selected from the group consisting of VCP, p130^{cas}, EGF receptor, p210 bcr:abl, MAP kinase, Shc, insulin receptor, lck and T cell receptor zeta chain.

6. The method of claim 1 wherein the substrate trapping mutant protein tyrosine phosphatase comprises a protein tyrosine phosphatase in which the wildtype protein tyrosine phosphatase catalytic domain invariant aspartate residue is replaced with an amino acid which does not cause significant alteration of the Km of the enzyme but which results in a reduction in Kcat to less than 1 per minute.

7. The method of claim 1 wherein the substrate trapping mutant protein tyrosine phosphatase comprises a protein tyrosine phosphatase in which the wildtype protein tyrosine phosphatase catalytic domain is mutated at an amino acid position occupied by a cysteine residue.

8. The method of claim 6 wherein the substrate trapping mutant protein tyrosine phosphatase comprises a protein tyrosine phosphatase in which at least one wildtype tyrosine residue is replaced with an amino acid that is not capable of being phosphorylated.

9. The method of claim 8 wherein at least one wildtype tyrosine residue is replaced with an amino acid selected from the group consisting of alanine, cysteine, aspartic acid, glutamine, glutamic acid, phenylalanine, glycine, histidine, isoleucine, lysine, leucine, methionine, asparagine, proline, arginine, valine and tryptophan.

10. The method of claim 8 wherein at least one tyrosine residue that is located in a protein tyrosine phosphatase catalytic domain is replaced.

11. The method of claim 8 wherein at least one tyrosine residue that is located in a protein tyrosine phosphatase active site is replaced.

12. The method of claim 8 wherein the wildtype tyrosine residue is replaced with phenylalanine.

13. The method of claim 8 wherein the wildtype tyrosine residue that is replaced is a protein tyrosine phosphatase conserved residue.

14. The method of claim 13 wherein the conserved residue corresponds to tyrosine at amino acid position 676 in human PTPH1.

15. The method of claim 8 wherein at least one tyrosine residue is replaced with an amino acid that stabilizes a complex comprising the protein tyrosine phosphatase and at least one substrate molecule.

16. The method of claim 8 wherein the substrate trapping mutant protein tyrosine phosphatase is a mutated protein tyrosine phosphatase selected from the group consisting of PTP1B, PTP-PEST, PTPγ, MKP-1, DEP-1, PTPμ, PTPX1, PTPX10, SHP2, PTP-PEZ, PTP-MEG1, LC-PTP, TC-PTP, CD45, LAR and PTPH1.

17. A method for identifying an agent which alters the interaction between a protein tyrosine phosphatase and a tyrosine phosphorylated polypeptide which is a substrate of the protein tyrosine phosphatase, comprising:
(a) contacting, in the absence and in the presence of a candidate agent, a protein tyrosine phosphatase and a detectably labeled tyrosine phosphorylated peptide which is a substrate of the protein tyrosine phosphatase under conditions and for a time sufficient to permit dephosphorylation of the substrate by the protein tyrosine phosphatase, wherein the substrate is capable of generating a fluorescence energy signal;
(b) exposing the protein tyrosine phosphatase and the substrate to a reaction terminator molecule and thereby terminating dephosphorylation of the substrate; and
(c) comparing the fluorescence energy signal level of substrate which remains phosphorylated in the absence of the agent to the energy signal level of substrate which remains phosphorylated in the presence of the agent, wherein a difference in the fluorescence energy signal level indicates the agent alters the interaction between the protein tyrosine phosphatase and the substrate.

18. A method of identifying an agent which alters the interaction between a protein tyrosine phosphatase and a tyrosine phosphorylated polypeptide which is a substrate of the protein tyrosine phosphatase, comprising:
(a) contacting, in the absence and in the presence of a candidate agent, a protein tyrosine phosphatase and a detectably labeled tyrosine phosphorylated peptide which is a substrate of the protein tyrosine phosphatase under conditions and for a time sufficient to permit dephosphorylation of the substrate by the protein tyrosine phosphatase, wherein the substrate is capable of generating a fluorescence energy signal;
(b) exposing the protein tyrosine phosphatase and the substrate to a reaction terminator molecule and thereby terminating dephosphorylation of the substrate; and
(c) comparing the fluorescence energy signal level of substrate which is dephosphorylated in the absence of the agent to the energy signal level of substrate which is dephosphorylated in the presence of the agent, wherein a difference in the fluorescence energy signal level indicates the agent alters the interaction between the protein tyrosine phosphatase and the substrate.

19. The method of either claim 17 or claim 18 wherein the fluorescence energy signal level is a fluorescence polarization signal level.

20. The method of either claim 17 or claim 18 wherein the detectably labeled tyrosine phosphorylated peptide substrate and the reaction terminator molecule comprise an energy transfer molecule donor-acceptor pair, and wherein the fluorescence energy signal level is a fluorescence resonance energy transfer level.

21. The method of claim 20 wherein the detectably labeled tyrosine phosphorylated peptide substrate comprises an energy transfer acceptor molecule and the reaction terminator molecule comprises an energy transfer donor molecule.

22. The method of claim 20 wherein the detectably labeled tyrosine phosphorylated peptide substrate comprises an energy transfer donor molecule and the reaction terminator molecule comprises an energy transfer acceptor molecule.

23. The method of either claim 17 or claim 18, wherein the reaction terminator molecule is an antibody specific for phosphotyrosine and wherein the step of exposing is subsequent to the step of contacting the PTP and the substrate.

24. The method of either claim 17 or claim 18, wherein the reaction terminator molecule is selected from the group consisting of an antibody specific for a PTP substrate, an antibody specific for a PTP catalytic domain, a substrate trapping mutant protein tyrosine phosphatase, vanadate, an SH2 domain polypeptide, an IRS1 PTB domain polypeptide, an she PH domain polypeptide and a non-antibody molecule that specifically binds to a tyrosine phosphorylated form of the detectable PTP substrate.

25. The method of claim 24 wherein the molecule that specifically binds to a tyrosine phosphorylated form of the detectable substrate is selected from the group consisting of an SH2 domain polypeptide and a PTP-PID domain polypeptide.

26. A method for determining dephosphorylation of a substrate by a protein tyrosine phosphatase, comprising:
contacting a protein tyrosine phosphatase and a detectable substrate of said protein tyrosine phosphatase under conditions and for a time sufficient to permit dephosphorylation of said detectable substrate by said protein tyrosine phosphatase to form a reaction mixture, wherein the detectable substrate comprises a detectably labeled tyrosine phosphorylated peptide which is a substrate of the protein tyrosine phosphatase;
exposing to said reaction mixture a reaction terminator molecule; and
determining the level of tyrosine phosphorylated substrate in the reaction mixture by detecting anisotropic motion of the substrate, and therefrom determining dephosphorylation of the detectable substrate by the protein tyrosine phosphatase.

27. A method for identifying an agent that regulates dephosphorylation of a detectable substrate by a protein tyrosine phosphatase, comprising:
contacting a protein tyrosine phosphatase and a detectable substrate of said protein tyrosine phosphatase under conditions and for a time sufficient to permit dephosphorylation of said detectable substrate by said protein tyrosine phosphatase in the presence of a candidate agent to form a first incomplete reaction mixture, and in the absence of a candidate agent to form a second incomplete reaction mixture, wherein the detectable substrate comprises a detectably labeled tyrosine phosphorylated peptide which is a substrate of the protein tyrosine phosphatase;
exposing to each of said first and second incomplete reaction mixtures a reaction terminator molecule to form a first complete reaction mixture and a second complete reaction mixture; and
comparing the level of tyrosine phosphorylated substrate in each of said first and second complete reaction mixtures by detecting anisotropic motion of the substrate, and therefrom identifying an agent that regulates dephosphorylation of the detectable substrate by the protein tyrosine phosphatase.

28. The method of either claim 26 or claim 27 wherein the reaction terminator molecule is an antibody specific for phosphotyrosine and wherein the step of exposing is subsequent to the step of contacting the PTP and the substrate.

29. The method of either claim 26 or claim 27 wherein the reaction terminator molecule is selected from the group consisting of an antibody specific for a PTP substrate, an antibody specific for a PTP catalytic domain, a substrate trapping mutant protein tyrosine phosphatase, vanadate, an SH2 domain polypeptide, an IRS1 PTB domain polypeptide, an shc PH domain polypeptide and a non-antibody molecule that specifically binds to a tyrosine phosphorylated form of the detectable PTP substrate.

30. The method of claim 29 wherein the molecule that specifically binds to a tyrosine phosphorylated form of the detectable substrate is selected from the group consisting of an SH2 domain polypeptide and a PTP-PID domain polypeptide.

31. The method of either claim 26 or claim 27 wherein the detectable substrate comprises a fluorophore and wherein anisotropic motion is detected by fluorescence polarization.

32. The method of claim 31 wherein the fluorophore is selected from the group consisting of fluorescein, rhodamine, Texas Red, AlexaFluor-594, AlexaFluor-488, Oregon Green, BODIPY-FL and Cy-5.

33. The method of either claim 26 or claim 27 wherein the substrate comprises a polypeptide sequence derived from a protein selected from the group consisting of VCP, p130^{cas}, EGF receptor, p210 bcr:abl, MAP kinase, She, insulin receptor, lck and T cell receptor zeta chain.
